(19) United States
(12) Patent Application Publication
(10) Pub. No.: US 2020/0299251 A1
(43) Pub. Date: Sep. 24, 2020

(54) FURAN SKELETON-CONTAINING IMINOGUANIDINE DERIVATIVE AS WELL AS PREPARATION AND APPLICATION THEREOF

(71) Applicant: SUN YAT-SEN UNIVERSITY, Guangzhou (CN)

(72) Inventors: Yong ZOU, GUANGZHOU (CN); Qianzhong ZHANG, GUANGZHOU (CN); Xianheng SONG, GUANGZHOU (CN); Chun CHEN, GUANGZHOU (CN); Xiang LUO, GUANGZHOU (CN)

(73) Assignee: SUN YAT-SEN UNIVERSITY

(21) Appl. No.: 16/882,491

(22) Filed: May 24, 2020

Related U.S. Application Data
(63) Continuation of application No. PCT/CN2018/117144, filed on Nov. 23, 2018.

(30) Foreign Application Priority Data
Nov. 24, 2017 (CN) 201711187713.3

(51) Int. Cl. C07D 307/52; B01D 53/14

(52) U.S. Cl. CPC C07D 307/52; B01D 2253/20; C07B 2200/13; B01D 53/1475

(57) ABSTRACT

The disclosure discloses a furan skeleton-containing iminoguanidine derivative, comprising 2,5-furan-bis(iminoguanidine) and acceptable salts of 2,5-furan-bis(iminoguanidine) as well as solvates thereof. The disclosure also discloses a preparation method of 2,5-furan-bis(iminoguanidine) and its use as an acidic gas absorbent and an anionic precipitant. The 2,5-furan-bis(iminoguanidine) of the disclosure can be conveniently regenerated and recycled alter absorbing acidic gases, is low in regeneration energy consumption and has reduced cost and improved efficiency. The 2,5-furan-bis(iminoguanidine) of the disclosure is simple in preparation, mild in reaction conditions, short in reaction time, high in yield and low in cost, and is easily prepared on large scale.

### CROSS-REFERENCE TO RELATED APPLICATIONS

This application is a continuation of International Patent Application No. PCT/CN2018/117144 with a filing date of Nov. 23, 2018, designating the United States, now pending, and further claims priority to Chinese Patent Application No. 201711187713.3 with a filing date of Nov. 24, 2017. The content of the aforementioned applications, including any intervening amendments thereto, are incorporated herein by reference.

### TECHNICAL FIELD

The disclosure relates to the fields of chemical industry and environment protection, and particularly to a furan skeleton-containing iminoguanidine derivative as well as preparation and application thereof.

### BACKGROUD OF THE PRESENT INVENTION

With the development of economy and society, greenhouse effect and air pollution caused by use of fossil raw materials has seriously threatened the environment on which human beings live, and global climate warming, greenhouse effect and environmental pollution are common challenges for sustainable development of various countries. Studies show that the content of carbon dioxide in the atmosphere is increasing due to human activities, the concentration of carbon dioxide has risen from 280ppm prior to industrial revolution to over 400ppm at present, thereby resulting in aggravating the greenhouse effect of atmosphere, raising the average earth surface temperature, warming the global climate and increasing occurrence frequency and strength of relevant natural hazards. Such global climate change has seriously threatened the survival and development of human society. It is urgent for emission reduction of greenhouse gases represented by CO₂.

The CO₂ capture, utilization and storage (CCUS) technology is a direct emission reduction technology, which is extremely important for stabilizing the concentration of carbon dioxide in the atmosphere. The most crucial and principal step is the capture technology of CO₂ (Chem. Rev. 2016, 116, 11840-11876; Technological Economy of Energy, 2010, 22(4), 21-26; Low Carbon World, 2013, 3(1), 30-33). In such the technologies, the chemical absorption method of CO₂ is an important and effective CO₂ capture method. Typical chemical absorbents include alkyl alkylol amine, hot potassium carbonate solution and the like. It absorbs by utilizing the property of CO₂ serving as an acidic gas and using an alkaline substance, then desorbs by heating so as to achieve the purpose for the concentrating and enriching of CO₂. However, this method has the disadvantages that the absorbent is large in regeneration energy consumption, easy in degradation, easy in volatilization, strong in equipment corrosion and the like.

Preparation of various chemicals (including fuels, basic chemical raw materials, fine chemicals and drugs) by replacing fossil resources with renewable resources is an important and representative low-carbon emission technology, which has been widely used in the fields of biomass energy and the like, and has produced good social and economic benefits (Environ. Sci. Technol., 2017, 51, 3575-3583; Angew. Chem. Int. Ed., 2007, 46, 5056-5058; Sino-Global Energy, 2014, 19, 21-26). If the design, synthesis, development and application of chemical absorbents for CO₂ are performed based on renewable resources, the preparation and application of the absorbent itself would not consume or would consume less fossil resources and would thus produce less carbon emissions, and would consequently, be highly beneficial for efficient CCUS. Meanwhile, the CO₂ capture technology is highly related to the capturing technologies of other acidic gases (such as SO₂, SO₃, NO₂ and H₂S) and anions (Angew. Chem. Int. Ed. 2015, 54, 10525-10529), and is expected to produce application values in many fields.

### SUMMARY OF PRESENT INVENTION

The object of the disclosure is to overcome the defects of the prior art, provide 2,5-furan-bis(iminoguanidine) (FuBIG for short, structure 1) which is environmental friendly, low in cost and simple in process and contain a furan structure unit, as well as acceptable salts thereof and solvates thereof, wherein the furan structure unit is derived from a renewable resource.

Another object of the disclosure is to provide a method for preparing the above 2,5-furan-bis(iminoguanidine).

Still another object of the disclosure is to provide use of the above 2,5-furan-bis(iminoguanidine) as an acidic gas absorbent.

Yet another object of the disclosure is to provide use of the above 2,5-furan-bis(iminoguanidine) as an anion precipitant.

Objects of the disclosure are realized by the following technical solution:

Provided is a 2,5-furan-bis(iminoguanidine) compound, which has the following structural formula:

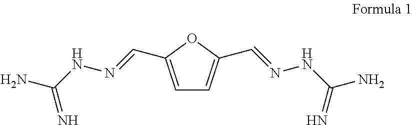 [SMILES: N=C(N)N/N=C/c1ccc(/C=N/NC(=N)N)o1]

The acceptable salts of the above 2,5-furan-bis(iminoguanidine) as well as solvates thereof comprise but are not limited to carbonate and solvates thereof, sulfite and solvates thereof, sulfide and solvates thereof, hydrochloride and solvates thereof, sulfate and solvates, nitrate and solvates thereof, phosphate and solvates thereof, pypocholoride and solvates thereof, perchlorate and solvates thereof, bichromate and solvates thereof, and permanganate and solvates thereof. The solvates comprise but are not limited to hydrates, methanol compounds and ethanol compounds.

Provided is a method for preparing the above 2,5-furan-bis(iminoguanidine), comprising the following steps: allowing 2,5-diformylfuran as the starting material to react with aminoguanidine hydrochloride in solvent A; after the reaction is ended, allowing the reaction solution to being subjected to standing at a certain temperature, and filtering to obtain 2,5-furan-bis(iminoguanidine) hydrochloride; and alkalizing and secondarily storing to obtain 2,5-furan-bis(iminoguanidine).

The 2,5-diformylfuran is prepared by using renewable biomass resource 5-hydroxymethylfurfural as the starting material (specifically see Chem. Rev. 2013, 113, 1499-1597).

The molar ratio of 2,5-diformylfuran to aminoguanidine hydrochloride is 1:11:3, preferably, the molar ratio is 1:2.

The molar ratio of 2,5-furan-bis(iminoguanidine) hydrochloride to alkali is 1:21:4, preferably, the molar ratio is 1:2.

The solvent A can be but not limited to methanol, ethanol, 1,4-dioxane or tetrahydrofuran; preferably, the solvent is methanol or ethanol.

The reaction temperature is 60˜100° C., preferably, the reaction temperature is 70° C.; the reaction time is 6˜24 h, preferably, the reaction time is 12 h.

The standing temperature of the reaction solution is 0˜40° C., preferably the temperature is 0˜10° C., and more preferably, the temperature is 4° C.; the standing time is 0.5˜12 h.

The alkali adopted by alkalizing can be but not limited to sodium hydroxide, potassium hydroxide, sodium carbonate, potassium carbonate or cesium carbonate.

The temperature of the secondary standing is 0˜40° C., preferably, the temperature is 0˜10° C., and more preferably, the temperature is 4° C.; the standing time is 0.5˜12 h.

Provided is use of the 2,5-furan-bis(iminoguanidine) as an acidic gas absorbent, wherein in solvent B, 2,5-furan-bis(iminoguanidine) is in contact with an acidic gas or a mixed gas containing the acidic gas to form a precipitate to be separated out, and filtered to obtain precipitate A. The precipitate A is a salt containing 2,5-furan-bis(iminoguanidine) and anions related to the acidic gas; this salt has extremely low liquid phase solubility. The precipitate A can release the acidic gas when being heated to a certain temperature and allows 2,5-furan-bis(iminoguanidine) to be regenerated. The released acidic gas can be collected, and the regenerated 2,5-furan-bis(iminoguanidine) can be in contact with the acidic gas or the mixed gas containing the acidic gas again, so as to repeat the above process. Such circulation can continuously enrich the acidic gas, and the enriched acidic gas can be released and recycled under mild conditions.

The acidic gas includes but is not limited to carbon dioxide, sulfur dioxide, sulfur trioxide, nitrogen dioxide, nitric oxide, nitrous oxide or hydrogen sulfide; the mixed gas containing the acidic gas includes but is not limited to a gas obtained by mixing the above acidic gases in any ratios and a gas obtained by mixing one or more acidic gases with air, nitrogen, oxygen or inert gas in any ratios.

The 2,5-furan-bis(iminoguanidine), as a carbon dioxide absorbent, is used for capture, utilization and storage of carbon dioxide.

The solvent B is one or a mixture of more of water, methanol, ethanol, acetone, tetrahydrofuran, acetonitrile, 1,4-dioxane, sulfolane, N-methylpyrrolidone, poly(glycol dimethyl ether) or propylene carbonate; preferably, the solvent B is one or a mixture of more of water, methanol or ethanol; more preferably, the solvent B is water, methanol, ethanol, 5˜95% (V/V) methanol aqueous solution or 5˜95% (V/V) ethanol aqueous solution.

The heating temperature of the precipitate A is 25˜180° C., preferably, the temperature is 40˜120° C., and more preferably, the temperature is 40˜100° C. .

Provided is use of the 2,5-furan-bis(iminoguanidine) derivative as an anion precipitant, wherein in solvent B, 2,5-furan-bis(iminoguanidine) generates a strong bonding effect with anion and forms a precipitate to be separated out; the anion forming the precipitate therewith includes but is not limited to carbonate, bicarbonate, sulfite, sulfite, sulfate, hydrogen sulfate, nitrate, hydrogen sulfide, phosphate, hydrogen phosphate, dihydrogen phosphate, perchlorate, hypochlorite, bichromate or permanganate.

In addition, a salt formed from 2,5-furan-bis(iminoguanidine) and the acidic gas in a water phase can be used as an efficient, controllable and energy-efficient acidic gas release agent. Specially, the salt formed from 2,5-furan-bis(iminoguanidine) and carbon dioxide in the water phase can be used as an efficient, controllable and low-energy consumption carbon dioxide gas release agent.

It is noted that since 2,5-furan-bis(iminoguanidine) has good absorption effect and binding capacity on acid gas, it can be loaded or dispersed on a carrier (including but not limited to activated carbon, chitosan, silica gel, macroporous adsorption resin, diatomite, organic framework materials, alumina, cyclodextrin, molecular sieve and zeolite) to form a solid phase absorbent which also has the effect and capacity of absorbing the acidic gases.

It is noted that CO₂ absorbed through 2,5-furan-bis(iminoguanidine) can be released by heating and used for chemical conversion, thereby achieving the effect and purpose of utilizing carbon dioxide. Examples of chemical reactions are as follows (specifically see Org. Lett., 2019, 21(7): 2013-2018):

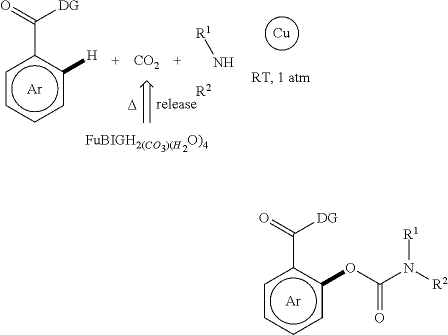

The process involved in the disclosure is as follows:

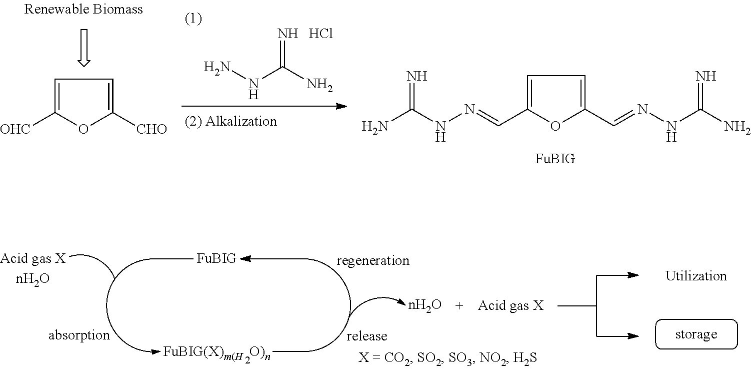 [SMILES: Cl ; O=Cc1ccc(C=O)o1 ; N=C(N)NN ; N=C(N)N/N=C/c1ccc(/C=N/NC(=N)N)o1]

Compared with the prior art, the disclosure has the following advantages and effects:

(1) the 2,5-furan-bis(iminoguanidine) of the disclosure is a new organic compound containing the furan skeleton, which has remarkable acidic gas absorption and anion bonding characteristics, and can be used in the fields of capturing, utilization and storage of carbon dioxide, air purification, pollution prevention, environmental protection and the like.

(2) The 2,5-furan-bis(iminoguanidine) of the disclosure can be easily regenerated and recycled after absorbing the acidic gas, with low regeneration energy consumption, thereby reducing the cost and improving the efficiency. The captured acidic gas can also be conveniently stored and recycled.

(3) The key raw material 2,5-furandialdehyde for preparing 2,5-furan-bis(iminoguanidine) in the disclosure is prepared from the renewable biomass resource 5-hydroxymethylfurfural (5-HMF) as the raw material. Therefore, the preparation method of the disclosure reduces the consumption of fossil resources, reduces carbon emissions, and is conducive to realizing sustainable development and application.

(4) The 2,5-furan-bis(iminoguanidine) of the disclosure is simple in preparation, mild in conditions, short in reaction time, with a high yield and low costs, and easily prepared on large scale.

### DESCRIPTION OF THE DRAWINGS

FIG. 1 is a ¹H nuclear magnetic resonance spectroscopy (400 MHz, DMSO-d₆) of 2,5-furan-bis(iminoguanidine) (FuBIG).

FIG. 2 is a nuclear ¹³C nuclear magnetic resonance spectroscopy (100 MHz, DMSO-d₆) of 2,5-furan-bis(iminoguanidine) (FuBIG).

FIG. 3 is a histogram of conversion rates of 2,5-furan-bis(iminoguanidine) (FuBIG) in example 32 via ten times of carbon dioxide absorption-release circulation.

FIG. 4 shows infrared spectrums of various substances collected through React IR in example 34.

FIG. 5 is a relative variation tendency chart illustrating mutual change between 2,5-furan-bis(iminoguanidine) and 2,5-furan-bis(iminoguanidine) carbonate tetrahydrate in example 34.

FIG. 6(A) and FIG. 6(B) are graphs illustrating mutual conversion between 2,5-furan-bis(iminoguanidine) and 2,5-furan-bis(iminoguanidine) carbonate tetrahydrate in example 34.

FIG. 7(A)-7(F) illustrate analytical result of X-ray single crystal diffraction of 2,5-furan-bis(iminoguanidine) carbonate tetrahydrate in example 35.

FIG. 8 is an X-ray powder diffraction graph of X-ray single crystal diffraction of 2,5-furan-bis(iminoguanidine) carbonate tetrahydrate in example 36.

FIG. 9(A) and FIG. 9(B) are TGA-IR test graphs of X-ray single crystal diffraction of 2,5-furan-bis(iminoguanidine) carbonate tetrahydrate in example 37, wherein FIG. 9(A) is the result of thermogravimetry analysis; FIG. 9(B) is the result of infrared test.

FIG. 10 illustrates curves of changes in weights of 2,5-furan-bis(iminoguanidine) carbonate tetrahydrate in example 38 respectively at 40, 50, 60, 70, 80, 90, 100 and 110° C. over time.

FIG. 11 is a function curve graph illustrating Jander model simulation of 80° C. constant-temperature thermogravimetry data in example 38.

FIG. 12 is a differential scanning calorimetry test graph of 2,5-furan-bis(iminoguanidine) carbonate tetrahydrate in example 39.

FIG. 13(A) and FIG. 13(B) illustrate test of solubility, FIG. 13(A) is an ultraviolet absorption spectrum of 5×10⁻⁵ M 2,5-furan-bis(iminoguanidine) hydrochloride aqueous solution in example 40, and FIG. 13(B) illustrates a standard ultraviolet absorption curve of 2,5-furan-bis(iminoguanidine) hydrochloride aqueous solution.

### DETAILED DESCRIPTION OF PREFERRED EMBODIMENTS

The disclosure will be further described in detail in combination with examples, however, embodiments of the disclosure are not limited thereto.

### Example 1: Preparation of 2,5-furan-bis(iminoguanidine)

2,5-furandialdehyde (12.4 g, 0.1 mol), aminoguanidine hydrochloride (22 g, 0.2 mol) and ethanol (100 ml) were added to a reaction bottle, stirred, heated to 70° C. and reacted for 12 h. After the reaction was ended, the reaction solution was subjected to standing for 12 h at 4° C. and then filtered at reduced pressure. The filter cake was washed with ethanol for three times. The filter cake was collected and dried to obtain 34.7 g of light yellow solid, which was 2,5-furan-bis(iminoguanidine) hydrochloride hydrate. The chemical formula was FuBIG.2HCl.2.5H₂O. Yield: 98.0%; mp: 184˜190° C.; elemental analysis for C₈H₁₉N₈O_{3.5}Cl₂, theoretical value: C: 27.13%; H: 5.41%; N: 31.64%; actually measured value: C: 26.62%; H: 5.08%; N:31.74%.

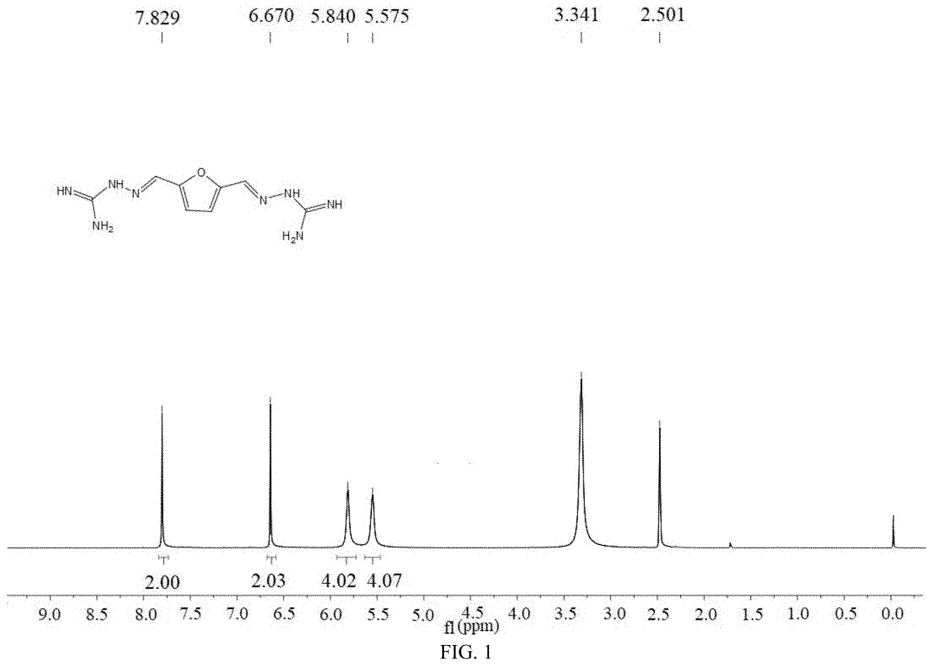

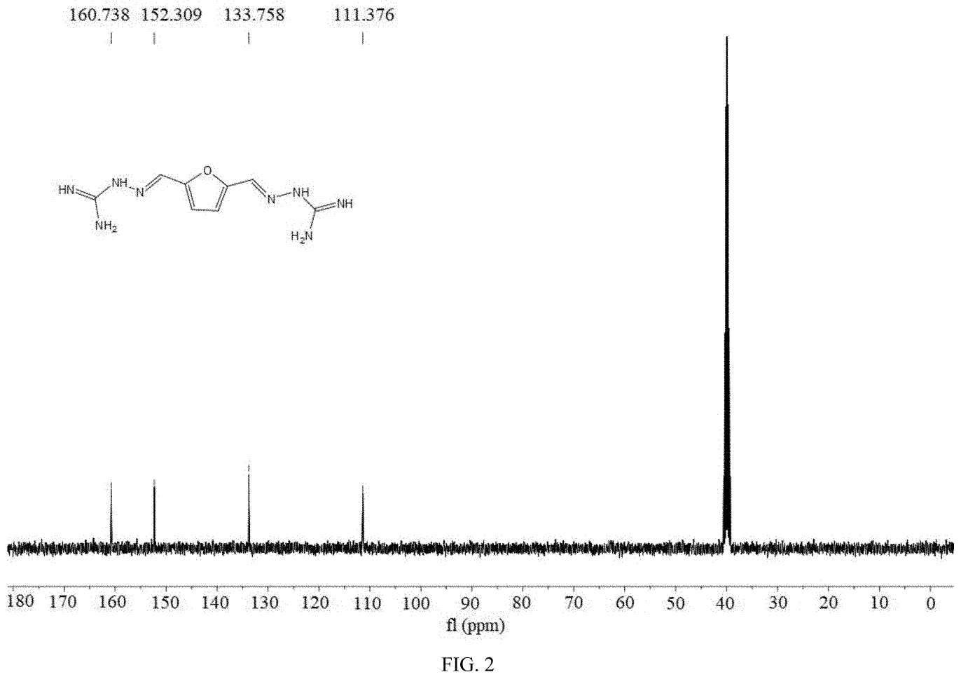

The above 2,5-furan-bis(iminoguanidine) hydrochloride hydrate was put in the reaction bottle, added with 100 ml of 2M sodium hydroxide aqueous solution, stirred for 0.5 h at room temperature, subjected to standing for 12 h at 4° C., filtered at reduced pressure and dried to obtain 22.66 g of 2,5-furan-bis(iminoguanidine). Yield: 96%, mp: 244-246° C. ¹HNMR (400 MHz, DMSO-d₆) δ 7.83 (s, 1H), 6.67 (s, 1H), 5.84 (s, 2H), 5.58 (s, 2H) (as shown in FIG. 1); ¹³C NMR (100 MHz, DMSO-d₆) δ 160.74, 152.31, 133.76, 111.38 (as shown in FIG. 2).

### Example 2: Preparation of 2,5-furan-bis(iminoguanidine)

2,5-furandialdehyde (12.4 g, 0.1 mol), aminoguanidine hydrochloride (22 g, 0.2 mol) and tetrahydrofuran (100 ml) were added to a reaction bottle, stirred, heated to 66° C. and reacted for 12 h. After the reaction was ended, the reaction solution was subjected to standing for 12 h at 4° C. and then filtered at reduced pressure. The filter cake was washed with tetrahydrofuran for three times. The filter cake was collected and dried to obtain a light yellow solid, which was 2,5-furan-bis(iminoguanidine) hydrochloride. 2,5-furyldiiminoguanidine hydrochloride was put in the reaction bottle, added with 100 ml of 2M sodium hydroxide aqueous solution, stirred for 0.5 h at room temperature, subjected to standing for 12 h at 4° C., filtered at reduced pressure and dried to obtain 21.72 g of 2,5-furan-bis(iminoguanidine). Yield: 92%, mp: 244-246° C.

### Example 3: Preparation of 2,5-furan-bis(iminoguanidine)

2,5-furandialdehyde (12.4 g, 0.1 mol), aminoguanidine hydrochloride (22 g, 0.2 mol) and methanol (100 ml) were added to a reaction bottle, stirred, heated to 65° C. and reacted for 12 h. After the reaction was ended, the reaction solution was subjected to standing at 4° C. for 6 h and then filtered at reduced pressure. The filter cake was washed with methanol for three times. The filter cake was collected and dried to obtain a light yellow solid, which was 2,5-furan-bis(iminoguanidine) hydrochloride. 2,5-furan-bis(iminoguanidine) hydrochloride was put in the reaction bottle, added with 100 ml of 2M sodium hydroxide aqueous solution, stirred for 0.5 h at room temperature, subjected to standing for 10 h at 4° C., filtered at reduced pressure and dried to obtain 21.24 g of 2,5-furyldiiminoguanidine. Yield: 90%, and mp: 244-246° C.

### Example 4: Preparation of 2,5-furan-bis(iminoguanidine)

2,5-furandialdehyde (12.4 g, 0.1 mol), aminoguanidine hydrochloride (22 g, 0.2 mol) and ethanol (100 ml) were added to a reaction bottle, stirred, heated to 60° C. and reacted for 10 h. After the reaction was ended, the reaction solution was subjected to standing for 6 h at 4° C. and then filtered at reduced pressure. A filter cake was washed with ethanol for three times. The filter cake was collected and dried to obtain a light yellow solid, which was 2,5-furan-bis(iminoguanidine) hydrochloride. 2,5-furan-bis(iminoguanidine) hydrochloride was put in the reaction bottle, added with 100 ml of 2M sodium hydroxide aqueous solution, stirred for 0.5 h at room temperature, subjected to standing for 6 h at 10° C., filtered at reduced pressure and dried to obtain 21.0 g of 2,5-furan-bis(iminoguanidine). Yield: 89%, and mp: 244-246° C.

### Example 5: Preparation of 2,5-furan-bis(iminoguanidine)

2,5-furandialdehyde (12.4 g, 0.1 mol), aminoguanidine hydrochloride (22 g, 0.2 mol) and 1,4-dioxane (100 ml) were added to a reaction bottle, stirred, heated to 80° C. and reacted for 12 h. After the reaction was ended, the reaction solution was subjected to standing for 12 h at 4° C. and then filtered at reduced pressure. A filter cake was washed with 1,4-dioxane for three times. The filter cake was collected and dried to obtain light yellow solid, which was 2,5-furan-bis(iminoguanidine) hydrochloride. 2,5-furan-bis(iminoguanidine) hydrochloride was put in the reaction bottle, added with 100 ml of 2M sodium carbonate aqueous solution, stirred for 0.5 h at room temperature, subjected to standing for 3 h at 30° C., filtered at reduced pressure and dried to obtain 21.24 g of 2,5-furan-bis(iminoguanidine). Yield: 90%, and mp: 244-246° C.

### Example 6: Preparation of 2,5-furan-bis(iminoguanidine)

2,5-furandialdehyde (12.4 g, 0.1 mol), aminoguanidine hydrochloride (22 g, 0.2 mol) and ethanol (100 ml) were added to a reaction bottle, stirred, heated to 70° C. and reacted for 6 h. After the reaction was ended, the reaction solution was subjected to standing for 12 h at 4° C. and then filtered at reduced pressure. A filter cake was washed with ethanol for three times. The filter cake was collected and dried to obtain light yellow solid, which was 2,5-furan-bis(iminoguanidine) hydrochloride. 2,5-furan-bis(iminoguanidine) hydrochloride was put in the reaction bottle, added with 100 ml of 2M potassium carbonate aqueous solution, stirred for 0.5 h at room temperature, subjected to standing for 12 h at 0° C., filtered at reduced pressure and dried to obtain 21.95 g of 2,5-furan-bis(iminoguanidine). Yield: 93%, and mp: 244-246° C.

### Example 7: Preparation of 2,5-furan-bis(iminoguanidine)

2,5-furandialdehyde (12.4 g, 0.1 mol), aminoguanidine hydrochloride (22 g, 0.2 mol) and ethanol (100 ml) were added to a reaction bottle, stirred, heated to 70° C. and reacted for 18 h. After the reaction was ended, the reaction solution was subjected to standing for 6 h at 4° C. and then filtered at reduced pressure. A filter cake was washed with ethanol for three times. The filter cake was collected and dried to obtain light yellow solid, which was 2,5-furan-bis(iminoguanidine) hydrochloride. 2,5-furan-bis(iminoguanidine) hydrochloride was put in the reaction bottle, added with 100 ml of 2M sodium hydroxide aqueous solution, stirred for 0.5 h at room temperature, subjected to standing for 1 h at 25° C., filtered at reduced pressure and dried to obtain 19.59 g of 2,5-furan-bis(iminoguanidine). Yield: 83%, and mp: 244-246° C.

### Example 8: Preparation of 2,5-furan-bis(iminoguanidine)

2,5-furandialdehyde (12.4 g, 0.1 mol), aminoguanidine hydrochloride (22 g, 0.2 mol) and ethanol (100 ml) were added to a reaction bottle, stirred, heated to 70° C. and reacted for 12 h. After the reaction was ended, the reaction solution was subjected to standing for 12 h at room temperature and then filtered at reduced pressure. A filter cake was washed with ethanol for three times. The filter cake was collected and dried to obtain light yellow solid, which was 2,5-furan-bis(iminoguanidine) hydrochloride. 2,5-furan-bis(iminoguanidine) hydrochloride was put in the reaction bottle, added with 100 ml of 2M sodium hydroxide aqueous solution, stirred for 0.5 h at room temperature, subjected to standing for 12 h at 4° C., filtered at reduced pressure and dried to obtain 20.53 g of 2,5-furan-bis(iminoguanidine). Yield: 87%, and mp: 244-246° C.

### Example 9: Preparation of 2,5-furan-bis(iminoguanidine)

2,5-furandialdehyde (12.4 g, 0.1 mol), aminoguanidine hydrochloride (22 g, 0.2 mol) and ethanol (100 ml) were added to a reaction bottle, stirred, heated to 70° C. and reacted for 12 h. After the reaction was ended, the reaction solution was subjected to standing for 6 h at 4° C. and then filtered at reduced pressure. A filter cake was washed with ethanol for three times. The filter cake was collected and dried to obtain light yellow solid, which was 2,5-furan-bis(iminoguanidine) hydrochloride. 2,5-furan-bis(iminoguanidine) hydrochloride was put in the reaction bottle, added with 100 ml of 2M sodium hydroxide aqueous solution, stirred for 0.5 h at room temperature, subjected to standing for 12 h at 4° C., filtered at reduced pressure and dried to obtain 20.06 g of 2,5-furan-bis(iminoguanidine). Yield: 85%, and mp: 244-246° C.

### Example 10: Preparation of 2,5-furan-bis(iminoguanidine)

2,5-furandialdehyde (12.4 g, 0.1 mol), aminoguanidine hydrochloride (22 g, 0.2 mol) and ethanol (100 ml) were added to a reaction bottle, stirred, heated to 70° C. and reacted for 12 h. After the reaction was ended, the reaction solution was subjected to standing for 12 h at 4° C. and then filtered at reduced pressure. A filter cake was washed with ethanol for three times. The filter cake was collected and dried to obtain light yellow solid, which was 2,5-furan-bis(iminoguanidine) hydrochloride. 2,5-furan-bis(iminoguanidine) hydrochloride was put in the reaction bottle, added with 100 ml of 2M potassium hydroxide aqueous solution, stirred for 0.5 h at room temperature, subjected to standing for 12 h at 4° C., filtered at reduced pressure and dried to obtain 20.48 g of 2,5-furan-bis(iminoguanidine). Yield: 91%, and mp: 244-246° C.

### Example 11: Preparation of 2,5-furan-bis(iminoguanidine)

2,5-furandialdehyde (12.4 g, 0.1 mol), aminoguanidine hydrochloride (22 g, 0.2 mol) and ethanol (100 ml) were added to a reaction bottle, stirred, heated to 70° C. and reacted for 12 h. After the reaction was ended, the reaction solution was subjected to standing for 12 h at 4° C. and then filtered at reduced pressure. A filter cake was washed with ethanol for three times. The filter cake was collected and dried to obtain light yellow solid, which was 2,5-furan-bis(iminoguanidine) hydrochloride. 2,5-furan-bis(iminoguanidine) hydrochloride was put in the reaction bottle, added with 100 ml of 2M sodium carbonate aqueous solution, stirred for 0.5 h at room temperature, subjected to standing for 12 h at 40° C., filtered at reduced pressure and dried to obtain 14.89 g of 2,5-furan-bis(iminoguanidine). Yield: 63%, and mp: 244-246° C.

### Example 12: Preparation of 2,5-furan-bis(iminoguanidine)

2,5-furandialdehyde (12.4 g, 0.1 mol), aminoguanidine hydrochloride (22 g, 0.2 mol) and ethanol (100 ml) were added to a reaction bottle, stirred, heated to 70° C. and reacted for 12 h. After the reaction was ended, the reaction solution was subjected to standing for 0.5 h at 4° C. and then filtered at reduced pressure. A filter cake was washed with ethanol for three times. The filter cake was collected and dried to obtain light yellow solid, which was 2,5-furan-bis(iminoguanidine) hydrochloride. 2,5-furan-bis(iminoguanidine) hydrochloride was put in the reaction bottle, added with 100 ml of 2M potassium carbonate aqueous solution, stirred for 0.5 h at room temperature, subjected to standing for 3 h at 4° C., filtered at reduced pressure and dried to obtain 15.58 g of 2,5-furan-bis(iminoguanidine). Yield: 66%, and mp: 244-246° C.

### Example 13: Absorption of 2,5-furan-bis(iminoguanidine) Aqueous Solution on Carbon Dioxide in Air

2,5-furan-bis(iminoguanidine) (2.36 g, 10 mmol) was dissolved into 200 ml of water and stirred for 3 h at room temperature under the condition of sufficiently contacting with air to separate out a yellow solid. The yellow solid was filtered at reduced pressure and dried to obtain 3.37 g of yellow powder, which was 2,5-furan-bis(iminoguanidine) carbonate tetrahydrate (FuBIGH₂(CO₃)(H₂O)₄). Yield: 91%.

### Example 14: Absorption of 2,5-furan-bis(iminoguanidine) Aqueous Solution on Carbon Dioxide in Air

2,5-furan-bis(iminoguanidine) (2.36 g, 10 mmol) was dissolved into 100 ml of water and stirred for 12 h at room temperature under the condition of sufficiently contacting with air to separate out a yellow solid. The yellow solid was filtered at reduced pressure and dried to obtain yellow powder, which was 2,5-furan-bis(iminoguanidine) carbonate tetrahydrate (FuBIGH₂(CO₃)(H₂O)₄). Weight: 3.52 g, and Yield: 95%.

### Example 15: Absorption of 50% Ethanol Aqueous Solution of 2,5-furan-bis(iminoguanidine) on Carbon Dioxide in Air

2,5-furan-bis(iminoguanidine) (2.36 g, 10 mmol) was dissolved into 100 ml of 50% ethanol aqueous solution and stirred for 12 h at room temperature under the condition of sufficiently contacting with air to separate out a yellow solid. The yellow solid was filtered at reduced pressure and dried to obtain yellow powder, which was 2,5-furan-bis(iminoguanidine) carbonate tetrahydrate (FuBIGH₂(CO₃)(H₂O)₄). Weight: 3.15 g, and Yield: 85%.

### Example 16: Absorption of 5% Methanol Aqueous Solution of 2,5-furan-bis(iminoguanidine) on Carbon Dioxide

2,5-furan-bis(iminoguanidine) (2.36 g, 10 mmol) was dissolved into 100 ml of 5% methanol aqueous solution and stirred for lh at room temperature under the condition of sufficiently contacting with air to separate out a yellow solid. The yellow solid was filtered at reduced pressure and dried to obtain yellow powder, which was 2,5-furan-bis(iminoguanidine) carbonate tetrahydrate (FuBIGH₂(CO₃)(H₂O)₄). Weight: 3.44 g, and Yield: 93%.

### Example 17: Absorption of 25% Methanol Aqueous Solution of 2,5-furan-bis(iminoguanidine) on Carbon Dioxide

2,5-furan-bis(iminoguanidine) (2.36 g, 10 mmol) was dissolved into 100 ml of 25% methanol aqueous solution and stirred for 12 h at room temperature under the condition of sufficiently contacting with air to separate out a yellow solid. The yellow solid was filtered at reduced pressure and dried to obtain yellow powder, which was 2,5-furan-bis(iminoguanidine) carbonate tetrahydrate (FuBIGH₂(CO₃)(H₂O)₄). Weight: 3.16 g, and Yield: 85%.

### Example 18: Absorption of 50% Methanol Aqueous Solution of 2,5-furan-bis(iminoguanidine) on Carbon Dioxide

2,5-furan-bis(iminoguanidine) (2.36 g, 10 mmol) was dissolved into 100 ml of 50% methanol water and stirred for lh at room temperature under the condition of sufficiently contacting with air to precipitate a yellow solid. The yellow solid was filtered at reduced pressure and dried to obtain yellow 2,5-furan-bis(iminoguanidine) carbonate tetrahydrate (FuBIGH₂(CO₃)(H₂O)₄) powder. Weight: 3.10 g, and Yield: 84%.

### Example 19: Absorption of 75% Methanol Aqueous Solution of 2,5-furan-bis(iminoguanidine) on Carbon Dioxide

2,5-furan-bis(iminoguanidine) (2.36 g, 10 mmol) was dissolved into 200 ml of 75% methanol aqueous solution and stirred for 12 h at room temperature under the condition of sufficiently contacting with air to precipitate a yellow solid. The yellow solid was filtered at reduced pressure and dried to obtain yellow 2,5-furan-bis(iminoguanidine) carbonate tetrahydrate (FuBIGH₂(CO₃)(H₂O)₄) powder. Weight: 3.12 g, and Yield: 84%.

### Example 20: Absorption of 95% Methanol Aqueous Solution of 2,5-furan-bis(iminoguanidine) on Carbon Dioxide

2,5-furan-bis(iminoguanidine) (2.36 g, 10 mmol) was dissolved into 250 ml of 95% methanol aqueous solution and stirred for 1 h at room temperature under the condition of sufficiently contacting with air to separate out a yellow solid. The yellow solid was filtered at reduced pressure and dried to obtain yellow 2,5-furan-bis(iminoguanidine) carbonate tetrahydrate (FuBIGH₂(CO₃)(H₂O)₄) powder. Weight: 3.33 g, and Yield: 90%.

### Example 21: Absorption of 2,5-furan-bis(iminoguanidine) Aqueous Solution on Sulfur Dioxide

2,5-furan-bis(iminoguanidine) (2.36 g, 10 mmol) was dissolved into 100 ml of water, sulfur dioxide gas was introduced, and the above substances were stirred for 1 h to separate out a yellow solid. The yellow solid was filtered at reduced pressure and dried to obtain yellow 2,5-furan-bis(iminoguanidine) sulfite powder. The chemical formula: FuBIGH₂(SO₃)(H₂O)₄. Weight: 3.55 g, and Yield: 91%.

### Example 22: Absorption of 2,5-furan-bis(iminoguanidine) Aqueous Solution on Sulfur Dioxide

2,5-furan-bis(iminoguanidine) (2.36 g, 10 mmol) was dissolved into 100 ml of water, hydrogen sulfide gas was introduced, and the above substances were stirred for 1 h to separate out a yellow solid. The yellow solid was filtered at reduced pressure and dried to obtain yellow 2,5-furan-bis(iminoguanidine) sulfide powder. The chemical formula: FuBIGH₂(H₂S)₂(H₂O)_{3.5}. Weight: 3.08 g, and Yield: 84%; mp.217-222V ; element analysis, the theoretical value: C:26.01; H:6.82; N:30.33; S:17.36; the actually measured value: C:26.32%; H:5.16%; N:30.11%; S:18.90%.

### Example 23: Absorption of 2,5-furan-bis(iminoguanidine) Aqueous Solution on Nitrogen Dioxide

2,5-furan-bis(iminoguanidine) (2.36 g, 10 mmol) was dissolved into 100 ml of water, nitrogen dioxide gas was introduced, and the above substances were stirred for 1 h to separate out a yellow solid. The yellow solid was filtered at reduced pressure and dried to obtain yellow 2,5-furan-bis(iminoguanidine) nitrate powder. The chemical formula: FuBIGH₂(NO₃)₂(H₂O)₂. Weight: 3.74 g, and Yield: 94%.

### Example 24: Absorption of 2,5-furan-bis(iminoguanidine) Aqueous Solution on Sulfite Ions

50 ml of 0.05M 2,5-furan-bis(iminoguanidine) hydrochloride aqueous solution and 50 ml of 0.05M anhydrous sodium sulfite aqueous solution were weighed, added into a reaction bottle, stirred for 2 h, filtered at reduced pressure and dried to obtain 0.94 g of yellow powder which was FuBIGH₂(SO₃)₂(H₂O)₄. Yield: 96%; mp.254-261° C.; element analysis, the theoretical value: C:24.61%; H:5.64%; N:28.72%; S:8.21%; the actually measured value: C:24.73%; H:5.18%; N:28.56%; S:8.20%.

### Example 25: Absorption of 2,5-furan-bis(iminoguanidine) Aqueous Solution on Sulfite Ions

50 ml of 0.05M 2,5-furan-bis(iminoguanidine) hydrochloride aqueous solution and 50 ml of 0.05M anhydrous sodium sulfite aqueous solution were weighed, added into a reaction bottle, stirred for 2 h, filtered at reduced pressure and dried to obtain 0.87 g of yellow powder which was FuBIGH₂(SO₃)₂(H₂O)₄. Yield: 94.0%.

### Example 26: Absorption of 2,5-furan-bis(iminoguanidine) Aqueous Solution on Nitrate Ions

50 ml of 0.05M 2,5-furan-bis(iminoguanidine) hydrochloride aqueous solution and 50 ml of 0.1M sodium nitrate aqueous solution were weighed, added into a reaction bottle, stirred for 2 h, filtered at reduced pressure and dried to obtain 0.90 g of white powder which was FuBIGH₂(NO₃)₂(H₂O)₂. Yield: 90.6%; mp.195-204° C.; element analysis, the theoretical value: C:24.12%; H:4.56%; N:35.17%; the actually measured value: C:24.23%; H:4.31%; N:34.74%.

### Example 27: Absorption of 2,5-furan-bis(iminoguanidine) Aqueous Solution on Hydrogen Phosphate Ions

50 ml of 0.05M 2,5-furan-bis(iminoguanidine) hydrochloride aqueous solution and 50 ml of 0.05M disodium hydrogen phosphate aqueous solution were weighed, added into a reaction bottle, stirred for 2 h, filtered at reduced pressure and dried to obtain 0.99 g of white powder which was FuBIGH₂(H₂PO₄)₂(H₂O). Yield: 88%; mp.188-197° C.; element analysis, the theoretical value: C:27.84%; H:5.26%; N:32.46%; the actually measured value: C:26.22%; H:5.76%; N:33.11%.

### Example 28: Release-Absorption Experiment of 2,5-furan-bis(iminoguanidine) Carbonate Tetrahydrate on Carbon Dioxide

2,5-furan-bis(iminoguanidine) carbonate tetrahydrate (FuBIGH₂(CO₃)(H₂O)₄, 3.7 g, 10 mmol) was placed in watch glass, and heated for 5 h at 100° C. under the normal pressure. The weight was 2.46 g, and weight loss was 33.6% (the theoretical weight loss of 2,5-furobiimidine carbonate tetrahydrate after losing H₂O and CO₂ to regenerate 2,5-furobiimidine was 36.22%). The weight-lost solid was dissolved into water, carbon dioxide gas was introduced, and the above substances were stirred for lh to separate out a yellow solid. The yellow solid was filtered at reduced pressure and dried to obtain 3.52 g of yellow 2,5-furan-bis(iminoguanidine) carbonate tetrahydrate powder.

This experiment indicates that 2,5-furan-bis(iminoguanidine) carbonate tetrahydrate can release H₂O and CO₂ via heating and allows 2,5-furan-bis(iminoguanidine) to be regenerated; the regenerated 2,5-furan-bis(iminoguanidine) can continue to absorb CO₂ and form 2,5-furan-bis(iminoguanidine) carbonate tetrahydrate.

### Example 29: Release-Absorption Cycle Experiment of 2,5-furan-bis(iminoguanidine) Carbonate Tetrahydrate on Carbon Dioxide

2,5-furan-bis(iminoguanidine) carbonate tetrahydrate (FuBIGH₂(CO₃)(H₂O)₄, 3.7 g, 10 mmol) was placed in watch glass and heated for 12 h at 70° C. under the normal pressure. The weight was 2.6 g, and weight loss was 29.7%. The weight-lost solid was dissolved into water, carbon dioxide gas was introduced, and the above substances were stirred for 1 h to separate out a yellow solid. The yellow solid was filtered at reduced pressure and dried to obtain 3.56 g of yellow 2,5-furan-bis(iminoguanidine) carbonate tetrahydrate powder.

### Example 30: Release-Absorption Cycle Experiment of 2,5-furan-bis(iminoguanidine) Carbonate Tetrahydrate on Carbon Dioxide

2,5-furan-bis(iminoguanidine) carbonate tetrahydrate (FuBIGH₂(CO₃)(H₂O)₄, 3.7 g, 10 mmol) was placed in watch glass and heated for 12 h at 120° C. under the normal pressure. The weight was 2.46 g, and weight loss was 33.7%. The weight-lost solid was dissolved into water, carbon dioxide gas was introduced, and the above substances were stirred for lh to separate out a yellow solid. The yellow solid was filtered at reduced pressure and dried to obtain 3.54 g of yellow 2,5-furan-bis(iminoguanidine) carbonate tetrahydrate powder.

### Example 31: Release-Absorption Cycle Experiment of 2,5-furan-bis(iminoguanidine) Carbonate Tetrahydrate on Carbon Dioxide

2,5-furan-bis(iminoguanidine) carbonate tetrahydrate (FuBIGH₂(CO₃)(H₂O)₄, 3.7 g, 10 mmol) was placed in watch glass and heated for 12 h at 180° C. under the normal pressure. The weight was 2.45 g, and weight loss was 33.7%. The weight-lost solid was dissolved into water, carbon dioxide gas was introduced, and the above substances were stirred for lh to separate out a yellow solid. The yellow solid was filtered at reduced pressure and dried to obtain 3.55 g of yellow 2,5-furan-bis(iminoguanidine) carbonate tetrahydrate powder.

### Example 32: Release-Absorption Cycle Experiment of 2,5-furan-bis(iminoguanidine) on Carbon Dioxide

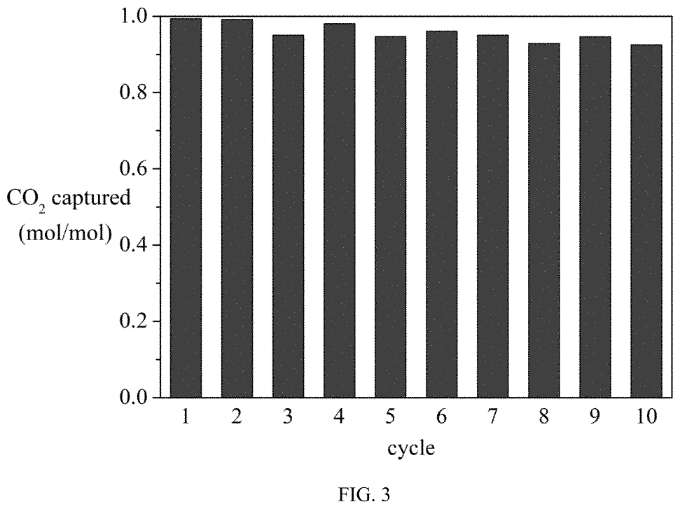

2,5-furan-bis(iminoguanidine) (FuBIG, 9.5 g, 0.04 mmol) was dissolved into 100 mL of water, the absorbance of 2,5-furan-bis(iminoguanidine) at 368 nm was measured to be substituted into the standard equation to calculate the concentration of 2,5-furan-bis(iminoguanidine) as 0.4248M; carbon dioxide gas was introduced, and the above substances were stirred for lh to separate out a yellow solid. The yellow solid was filtered at reduced pressure, the absorbance of filtrate at 368 nm was measured to be substituted into the standard equation to calculate the concentration of remaining 2,5-furan-bis(iminoguanidine) as 0.002713 M, and the conversion rate was 99.36%. The yellow solid was placed in watch glass and heated and dried for 12 h at 120° C. under the normal pressure. The weight-lost solid was dissolved into water again, the absorbance of the solid at 368 nm was measured, carbon dioxide gas was introduced again, and the above substances were stirred for lh to separate out a yellow solid. The yellow solid was filtered at reduced pressure, and the absorbance of filtrate at 368 nm was measured. After 2,5-furan-bis(iminoguanidine) was subjected to absorption-release cycle for ten times according to the same method, the conversion rate was 92.49%, as shown in FIG. 3.

### Example 33: Release of Carbon Dioxide and Chemical Conversion Involved—Synthesis of 2-(8-quinolinylaminoformyl) phenyl diethylcarbamate

1 g (4 mmol) of benzoylaminoquinoline, 827 μL (8 mmol) of diethylamine, 1.79 mL (12 mmol) of DBU, 0.15 g (0.8 mmol) of cupper iodide and 0.70 g (8 mmol) of manganese dioxide were weighed, and dissolved into a reaction vessel with 20 mL of DMF; 2.22 g (6 mmol) of 2,5-furan-bis(iminoguanidine) carbonate tetrahydrate was weighed in another reaction vessel. The above devices were connected via a breather pipe, 2,5-furan-bis(iminoguanidine) carbonate tetrahydrate was heated at 120° C. to be decomposed to release carbon dioxide. Without other carbon dioxide sources, the carbon-hydrogen activation reaction can also be smoothly carried out to obtain 2-(8-quinolinylaminoformyl) phenyl diethylcarbamate. The weight was 1.02 g, and Yield was 70%. mp: 85.3-86.1° C. ¹H NMR (400 MHz, CDCl₃) δ 10.66 (s, 1H), 8.96 (dd, J=7.8, 1.2 Hz, 1H), 8.80 (dd, J=4.0, 1.2 Hz, 1H), 8.24 (dd, J=8.4, 1.2 Hz, 1H), 8.00 (dd, J=7.6, 1.6 Hz, 1H), 7.62 (t, J=8.0 Hz, 1H), 7.57 (dd, J=8.0, 1.2 Hz, 1H), 7.55-7.46 (m, 2H), 7.35 (td, J=7.6, 0.8 Hz, 1H), 7.24 (dd, J=7.6, 0.8 Hz, 1H), 3.47 (q, J=7.2 Hz, 2H), 3.30 (q, J=7.2 Hz, 2H), 1.11 (t, J=7.2 Hz, 3H), 0.99 (t, J=7.2 Hz, 3H); 13C NMR (100 MHz, CDCl3) δ 164.29, 153.53, 149.05, 147.87, 138.52, 136.56, 134.81, 131.96, 130.28, 129.12, 128.01, 127.61, 125.58, 123.23, 121.80, 121.58, 117.24, 42.40, 42.02, 14.16, 13.11. HRMS (ESI) m/z [M+H]⁺: calcd for C₂₁H₂₁N₃O₃: 364.1656, found: 364.1658.

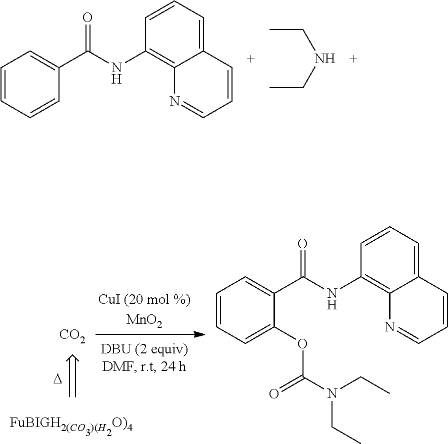

This experiment proves that the salt formed after 2,5-furan-bis(iminoguanidine) captures carbon dioxide and the solvate thereof (2,5-furan-bis(iminoguanidine) carbonate tetrahydrate) can be released under the mild condition and used for relevant chemical reactions.

The carbon dioxide recycle method is not limited to this example, and can be widely applied to other chemical reactions which carbon dioxide participates in.

### Example 34: Dynamic Experiment for Absorbing Carbon Dioxide by 2,5-furan-bis(iminoguanidine) Aqueous Solution

Experiment instrument: METTLER-TOLEDO on-line infrared analyzer (ReactlR 15)

Operation steps:

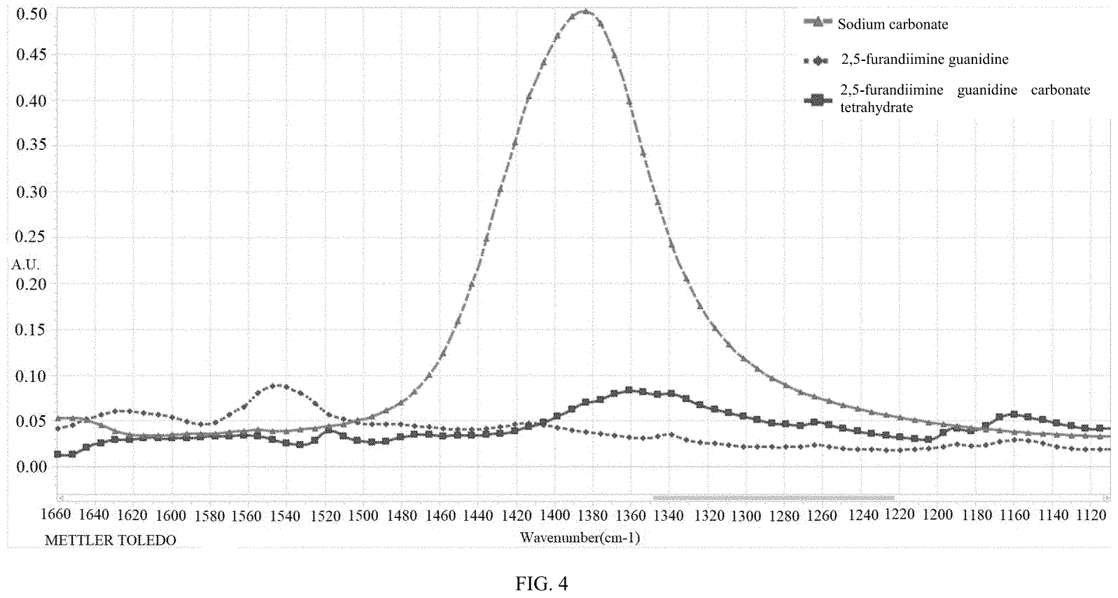

(1) The infrared spectrum of each substance was collected through the on-line infrared analyzer, as shown in FIG. 4. It can be seen from the spectrum of each substance that 2,5-furan-bis(iminoguanidine) and 2,5-furan-bis(iminoguanidine) carbonate tetrahydrate formed after 2,5-furan-bis(iminoguanidine) absorbs carbon dioxide have minimum overlap at 1511-1655 cm⁻¹ and 1280-1400 cm⁻¹ (namely carbonate absorption wavebands), thus 1533 cm⁻¹ is selected to track the change of 2,5-furan-bis(iminoguanidine), and 1361 cm⁻¹ is selected to track the change of 2,5-furan-bis(iminoguanidine) carbonate tetrahydrate.

(2) The cycle conversion process of 2,5-furan-bis(iminoguanidine) and 2,5-furan-bis(iminoguanidine) carbonate tetrahydrate was monitored using the on-line infrared analyzer.

A 100 ml double-mouth flask was placed into oil bath and magnetically stirred, and one mouth was inserted into an on-line infrared Dicomp probe and fixed with a Teflon adapter. After collecting the experimental background, 40 ml of 42 mm 2,5-furan-bis(iminoguanidine) aqueous solution was added into the flask and stirred and React IR data collection started (one data is collected every 0.5 min). After the data is stabilized, a carbon dioxide balloon was inserted into the other mouth of the flask, and continued to stir until 2,5-furan-bis(iminoguanidine) was completely converted into 2,5-furan-bis(iminoguanidine) carbonate tetrahydrate. Heating was started, and the temperature was gradually raised to 40° C., 50° C., 60° C. and 70° C., and the change of substances in the reaction bottle was observed.

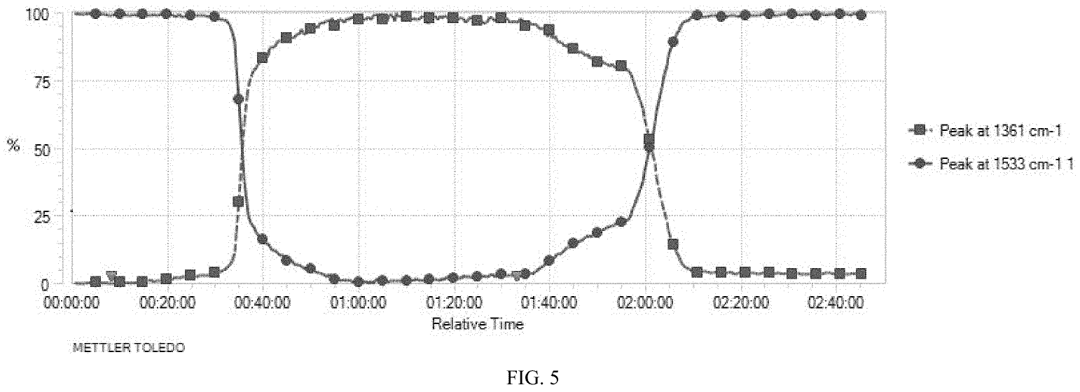

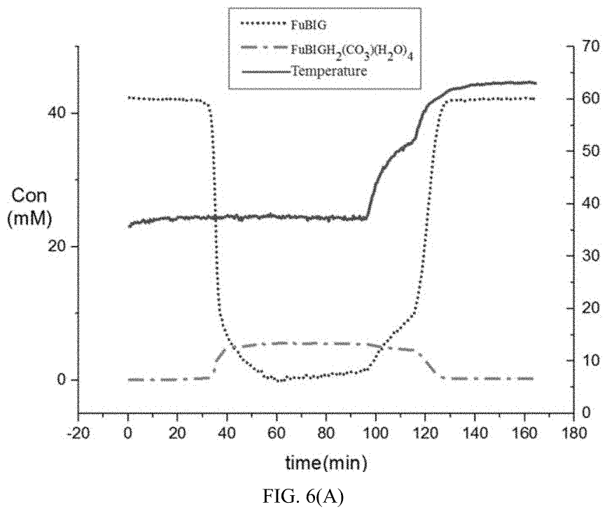

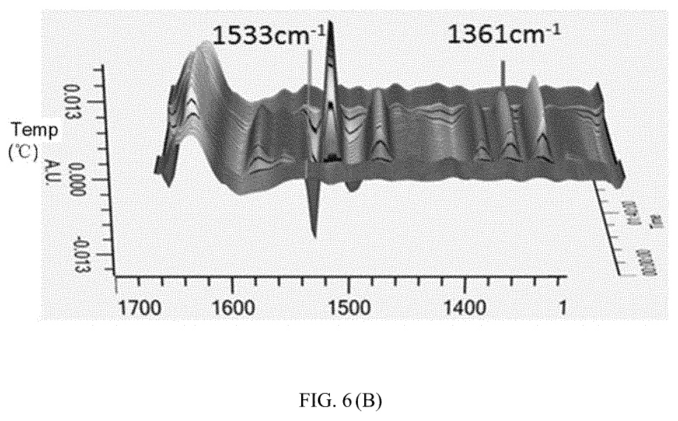

Data analysis: as shown in FIG. 5 and FIG. 6, 1361 cm⁻¹ is selected to track the change of 2,5-furan-bis(iminoguanidine) tetrahydrate, and 1533 cm⁻¹ is selected to track the change of 2,5-furan-bis(iminoguanidine). From the relative change tendency chart of 1361 cm⁻¹ and 1533 cm⁻¹, it can be seen that after CO₂ is introduced into the flask, the concentration of CO₃²⁻ in the reaction solution increases at a faster rate, while the concentration of 2,5-furan-bis(iminoguanidine) decreases at a corresponding rate. After about 1.5 h, the absorption of 2,5-furan-bis(iminoguanidine) aqueous solution on CO₂ reaches saturation, and a large number of solid (namely 2,5-furan-bis(iminoguanidine) carbonate tetrahydrate) is separated from the solution. When the precipitation is complete, heating is started. With the increase of temperature, 2,5-furan-bis(iminoguanidine) carbonate tetrahydrate is gradually converted into 2,5-furan-bis(iminoguanidine) and CO₂ is released, and the conversion rate is gradually accelerated with the increase of temperature. According to the data of on-line infrared analysis, heating the 2,5-furan-bis(iminoguanidine) carbonate tetrahydrate is capable of being quickly transformed into 2,5-furan-bis(iminoguanidine); the data shows that the rate of converting the 2,5-furan-bis(iminoguanidine) carbonate tetrahydrate into 2,5-furobiimidine reaches the maximum value at 55° C.

### Example 35: Preparation of 2,5-furan-bis(iminoguanidine) Carbonate Tetrahydrate Single Crystal and X-ray Diffraction Analysis

Preparation of signal crystal: 10 ml of 0.01M 2,5-furan-bis(iminoguanidine) aqueous solution was added into a round-bottom flask, and stood for one week at room temperature under the condition of opening to separate out the crystal, and the crystal was filtered to obtain yellow signal crystal.

Single crystal X-ray diffraction analysis: the crystal having a proper size and a good crystal form was selected to adhere to a glass fiber for determination of single crystal structure with a X-ray single crystal diffractometer (Xcalibur Nova). 12423 diffraction points and 5860 independent diffraction points (Rᵢₙₜ=0.0204, R_{sigma}=0.0213) were collected within the range of 2θₘₐₓ=134° at T=100K using Cu—Kα ray (λ=1.54184 Å) was used as a light source. The crystal structure was analyzed by XS (Sheldrick, 2008) and refined by SHELXL (Sheldrick, 2015). It is confirmed that the single crystal is 2,5-furan-bis(iminoguanidine) carbonate tetrahydrate with the chemical formula being FUBIGH₂ (CO₃)(H₂O)₄. The crystal test parameters are shown in Table 1.

TABLE 1
Crystal test parameters of 2,5-furan-bis(iminoguanidine)
carbonate tetrahydrate

| Chemical Formula | C₉H₂₂N₈O₈ |
|---|---|
| Formula Weight | 370.34 |
| Crystal Size (Mm³) | 0.5 × 0.3 × 0.2 |
| Crystal System | triclinic |
| Space Group | P-1 |
| a (Å) | 11.1919 (4) |
| b (Å) | 12.7930 (5) |
| c (Å) | 14.1784 (4) |
| α (deg) | 97.547 (3) |
| β (deg) | 111.174 (3) |
| γ (deg) | 112.709 (4) |
| volume (Å³) | 1657.46 (11) |
| Z | 4 |
| density (g/cm³) | 1.484 |
| 2θ range | 7.044-134.138 |
| F (000) | 787.0 |
| index ranges | −13 ≤ h ≤ 13 |
| | −15 ≤ k ≤ 15 |
| | −11 ≤ l ≤ 16 |
| no. of reflns | 11953 |
| no. of unique reflns | 5860 |
| no. of params | 501 |
| Rₐₗₗ, R_{obs}^{a} | 0.0352, 0.0336 |
| wR_{2,all}, wR_{2,obs}^{a} | 0.0892, 0.0880 |
| goodness-of-fit on F² | 1.054 |

^{a}R₁ = Σ||Fₒ| − |F_{c}||/Σ|Fₒ|, wR² = [Σ[w(Fₒ² − F_{c}²)² ] /Σw(Fₒ²)²]^{1/2}, w = 1/[σ²(Fₒ)² + (aP²) + bP], where P =[(Fₒ²) + 2F_{c}²]/3.

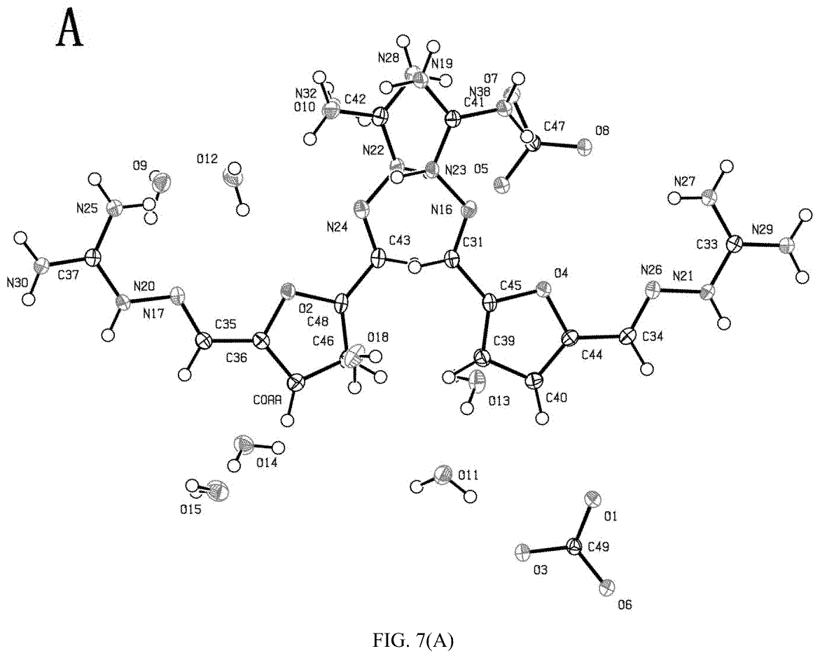

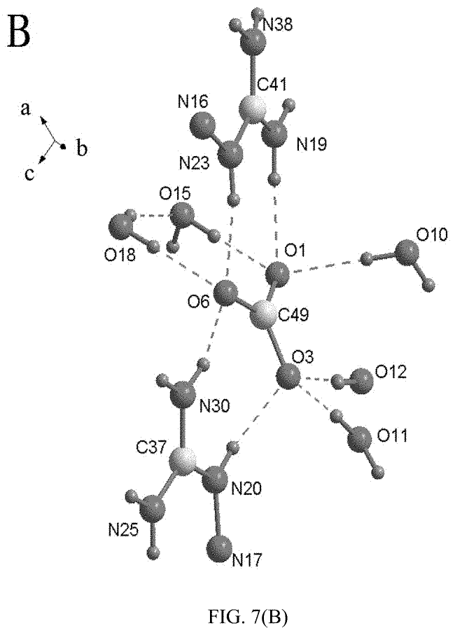

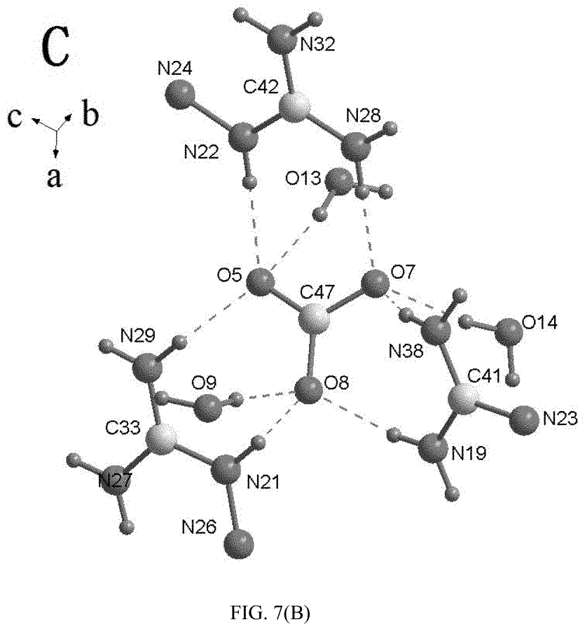

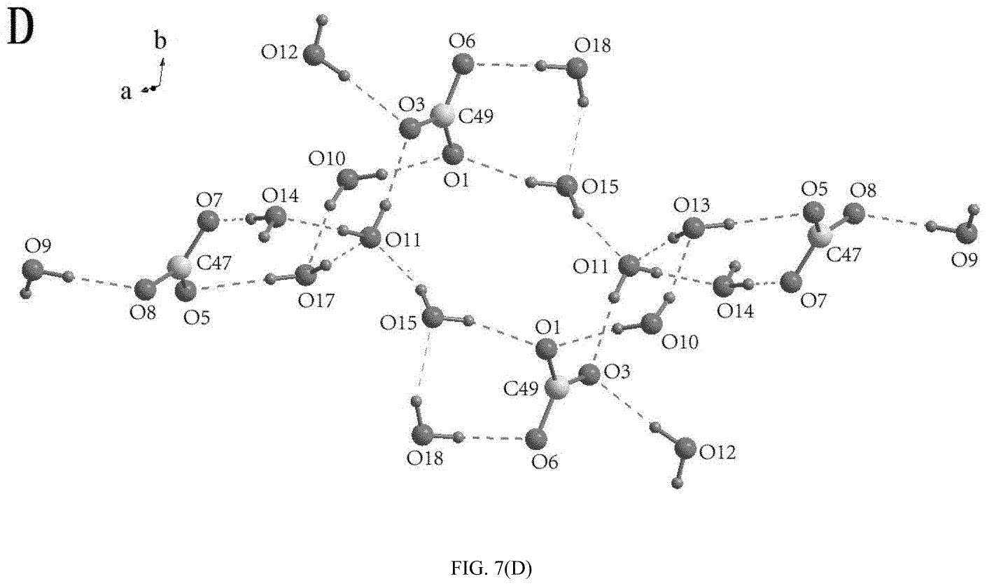

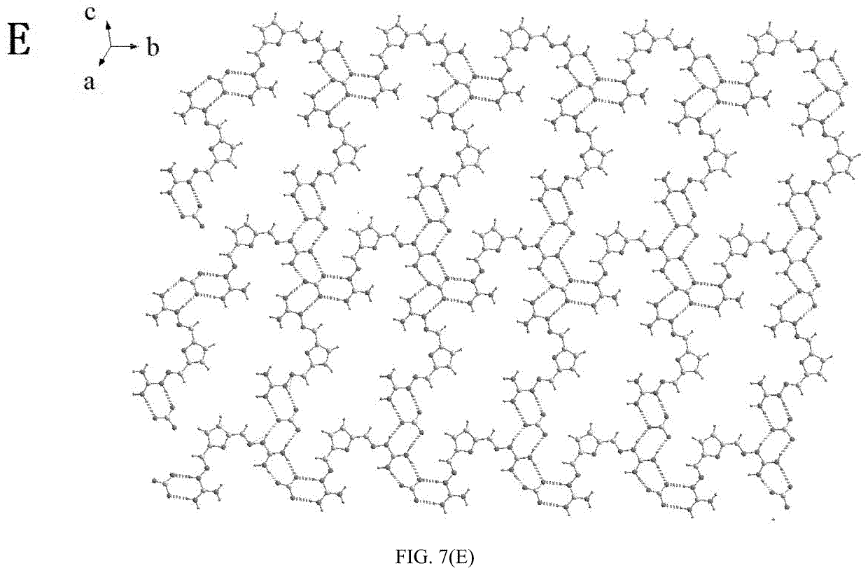

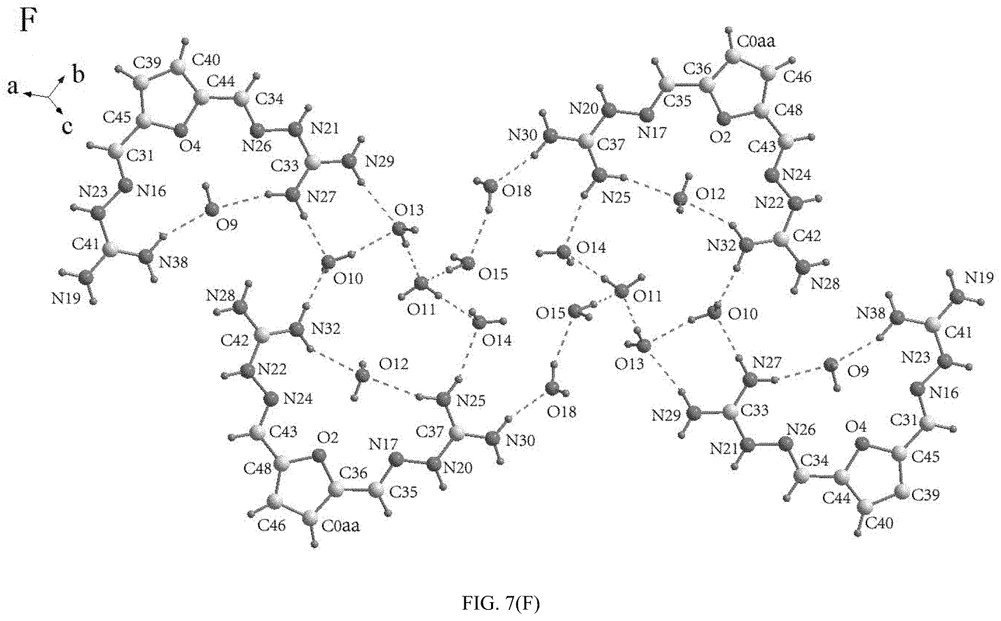

As shown in FIG. 7:

(1) A represents the molecular structure and chemical composition of 2,5-furan-bis(iminoguanidine) carbonate tetrahydrate (FUBIGH₂ (CO₃)(H₂O)₄) crystal which is constructed by one 2,5-furan-bis(iminoguanidine) carbonate (FUBIGH₂CO₃) and four water molecules via hydrogen bond action;

(2) B represents the first hydrogen bond action mode of CO₃²⁻ in the 2,5-furan-bis(iminoguanidine) carbonate tetrahydrate (FUBIGH₂CO₃(H₂O)₄) crystal: CO₃²⁻, as a hydrogen bond receptor, receives 9 hydrogen bonds, among which, five hydrogen bonds are from water molecules and 4 hydrogen bonds are from guanidino;

(3) C represents the second hydrogen bond action mode of CO₃²⁻ in 2,5-furan-bis(iminoguanidine) carbonate tetrahydrate (FUBIGH₂CO₃(H₂O)₄) crystal: CO₃²⁻, as a hydrogen bond receptor, receives 9 hydrogen bonds, among which, five hydrogen bonds are from water molecules and 6 hydrogen bonds are from guanidino;

(4) D represents the smallest complete unit formed by the hydrogen bond between CO₃²⁻ and guanidino in 2,5-furan-bis(iminoguanidine) carbonate tetrahydrate (FUBIGH₂CO₃(H₂O)₄) crystal via hydrogen bond action;

(5) E represents a super-molecular plane structure formed by the hydrogen bond between CO₃²⁻ and guanidino in 2,5-furan-bis(iminoguanidine) carbonate tetrahydrate (FUBIGH₂CO₃(H₂O)₄) crystal via hydrogen bond action;

(6) F represents the super-molecular structure formed by CO₃²⁻ and guanidino in 2,5-furan-bis(iminoguanidine) carbonate tetrahydrate (FUBIGH₂CO₃(H₂O)₄) crystal via hydrogen bond action.

The above experiments show that 2,5-furan-bis(iminoguanidine) can form complex hydrogen bond interaction with carbonate and water molecules, 2,5-furobiminoguanidine is only used as the donor of hydrogen bonds, CO₃²⁻ is only used as the receptor of the hydrogen bond. Each CO₃²⁻, as the receptor of the hydrogen bond, receives 9 hydrogen bonds. There are two hydrogen bond receiving modes (mode 1: in 9 hydrogen bonds, 5 donors are from water molecules, and 4 donors are from guanidino; mode 2: in 9 hydrogen bonds, 3 donors are from water molecules and 6 donors are from guanidino). The water molecule acts as both the donor of the hydrogen bond and the receptor of the hydrogen bond. The oxygen atom on a furan ring does not receive the hydrogen bond.

### Example 36: Powder Diffraction Experiment of 2,5-furan-bis(iminoguanidine) Carbonate Tetrahydrate (FuBIGH₂(CO₃)(H₂O)₄)

Experiment instrument and conditions: Panacean (Empyrean) X-ray diffractometer, Cu—Kα radiation, scanning range: 5°-80°, scanning speed: 5°/min, and step size: 0.02°. The single crystal sample obtained from example 33 was ground into powder for experiment.

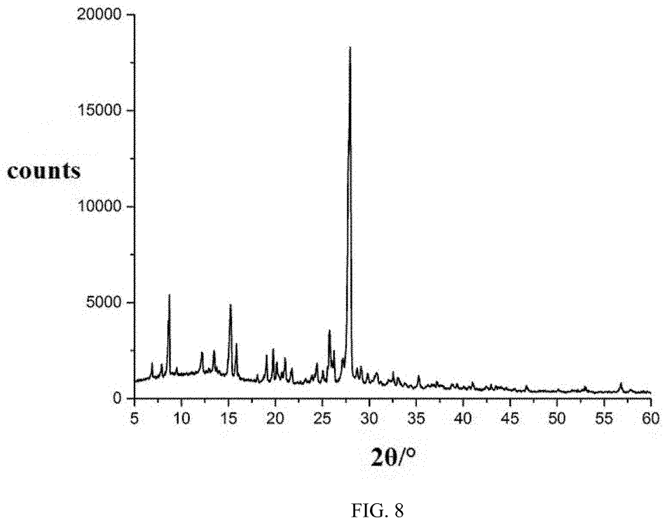

FIG. 8 is an X-ray powder diffraction graph of FuBIGH₂(CO₃)(H₂O)₄. X-ray powder diffraction represented by 2θ angle and interplanar spacing (d value) has characteristic peaks at about 6.85 (12.9), 7.87 (11.2), 8.67 (10.2), 13.48 (6.6), 15.27 (5.8), 15.87 (5.6), 19.05 (4.7), 19.77 (4.5), 21.06 (4.2), 24.45 (3.6), 25.75 (3.5), 27.75 (3.5) and 27.98 (3.2), the 2θ angle allows error of ±0.2°. The specific data is shown in Table 2:

TABLE 2
X-ray powder diffraction data of FuBIGH₂(CO₃)(H₂O)₄

| | Interplanar spacing | Relative intensity |
|---|---|---|
| 2θ(°) | (A) | (%) |
| 6.85 | 12.90 | 4.39 |
| 7.87 | 11.23 | 4.97 |
| 8.67 | 10.20 | 20.86 |
| 9.47 | 9.34 | 3.87 |
| 12.22 | 7.24 | 9.03 |
| 13.48 | 6.57 | 9.82 |
| 15.27 | 5.80 | 22.63 |
| 15.87 | 5.59 | 12.00 |
| 19.05 | 4.66 | 7.19 |
| 19.78 | 4.49 | 10.37 |
| 21.06 | 4.22 | 7.10 |
| 21.78 | 4.08 | 4.41 |
| 24.45 | 3.64 | 5.84 |
| 25.75 | 3.46 | 15.89 |
| 27.76 | 3.21 | 71.16 |
| 27.98 | 3.19 | 100.00 |
| 28.70 | 3.11 | 5.79 |
| 29.14 | 3.06 | 5.72 |
| 29.82 | 3.00 | 4.00 |
| 30.88 | 2.90 | 3.94 |

### Example 37: Thermogravimetry Infrared Experiment of 2,5-furan-bis(iminoguanidine) Carbonate Tetrahydrate (FuBIGH₂(CO₃)(H₂O)₄)

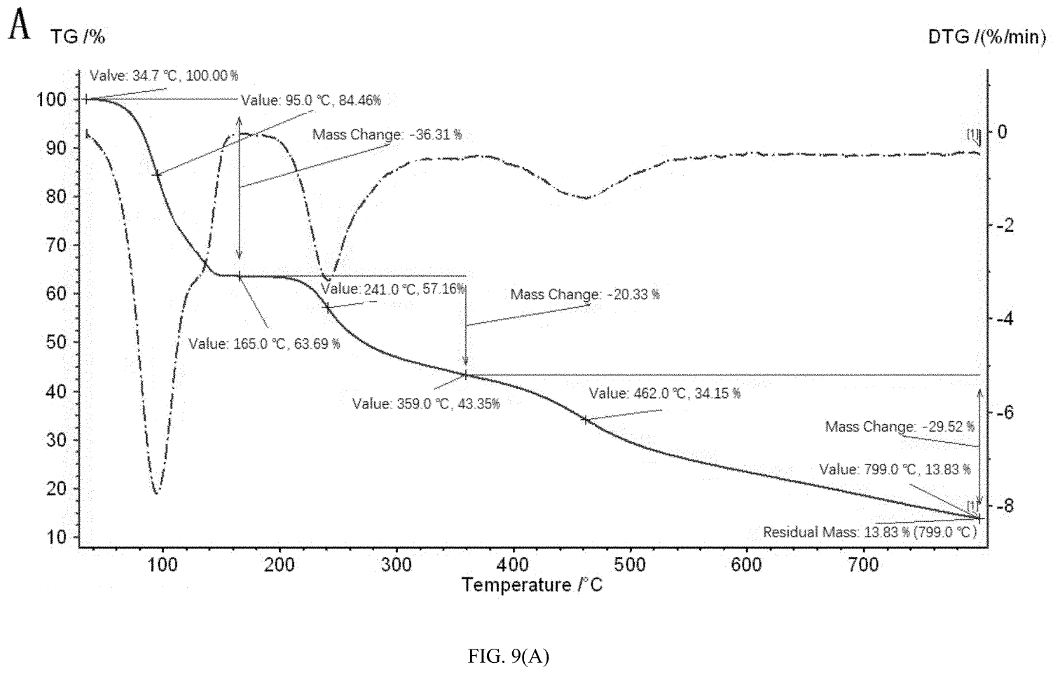

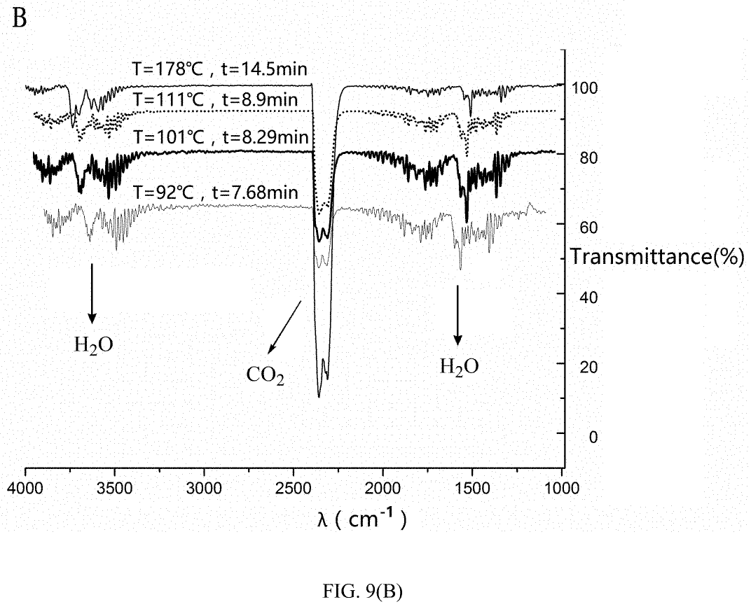

Thermogravimetry infrared analysis experiment was carried out on 2,5-furan-bis(iminoguanidine) carbonate tetrahydrate. The temperature range was 25-800° C., and the heating rate was 10 K/min. As shown in FIG. 9, the green line in FIG. A is the thermogravimetric curve of 2,5-furan-bis(iminoguanidine) carbonate tetrahydrate. The theoretical weight loss of 2,5-furan-bis(iminoguanidine) carbonate tetrahydrate (FUBIGH₂(CO₃)(H₂O)₄) after losing H₂O and CO₂ to regenerate 2,5-furan-bis(iminoguanidine) is 36.22%. It can be seen from FIG. 9A that the weight loss rate value of 2,5-furan-bis(iminoguanidine) carbonate tetrahydrate (FUBIGH₂(CO₃)(H₂O)₄) at 100° C. is the maximum, and water and carbon dioxide molecules completely lose weights at 165° C. with the weight loss reaching 36.31%. FIG. 9B is an infrared spectrum of 2,5-furan-bis(iminoguanidine) carbonate tetrahydrate (FUBIGH₂(CO₃)(H₂O)₄) eutectic compound varying with time and temperature at nitrogen atmosphere. It can be seen from FIG. 9B that this molecule can release water molecules and carbon dioxide molecules under the heating condition, and water molecules and carbon dioxide molecules are nearly released at the same time.

The above experiment shows that 2,5-furan-bis(iminoguanidine) carbonate tetrahydrate extremely easily releases H₂O and CO₂. For calcium carbonate, it releases CO₂ only if being roasted at a high temperature of more than 900° C. The method of the disclosure can greatly reduce the energy consumption of releasing CO₂.

### Example 38: Constant-Temperature Thermogravimetric Experiment and Analysis of 2,5-furan-bis(iminoguanidine) Carbonate Tetrahydrate (FUBIGH₂(CO₃)(H₂O)₄)

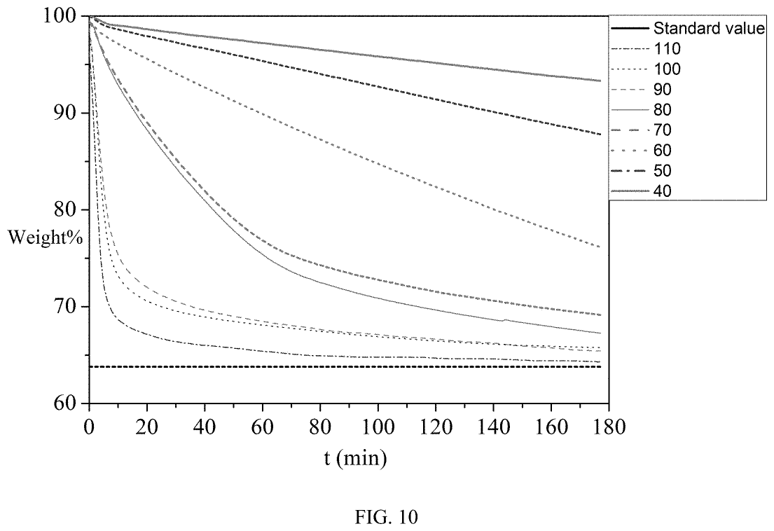

The constant-temperature thermogravimetric analysis experiment was carried out on 2,5-furan-bis(iminoguanidine) carbonate tetrahydrate. Change relationships of weights of 2,5-furan-bis(iminoguanidine) carbonate tetrahydrate at 40° C., 50° C., 60° C., 70° C., 80° C., 90° C., 100° C. and 110° C. over time were tested. As shown in FIG. 10, when the temperature exceeds 70° C. , 2,5-furan-bis(iminoguanidine) carbonate tetrahydrate (FUBIGH₂(CO₃)(H₂O)₄) loses H₂O and CO₂ to regenerate 2,5-furan-bis(iminoguanidine); the theoretical weight loss is 36.22%, as shown in the black line in the drawing. The loss rates of H₂O and CO₂ are different at different temperatures. The higher the temperature is, the faster the loss rate is, and the shorter the time to reach equilibrium is. When the temperature is higher than 100° C., the weight loss is close to the theoretical value.

The above experiments show that 2,5-furan-bis(iminoguanidine) carbonate tetrahydrate can release H₂O and CO₂ in a short time and at a low temperature.

The kinetic analysis was carried out to constant-temperature thermogravimetric data of 80° C. and above. The physical and chemical model screening of conversion fraction (a) and time t was carried out. It is found that release of CO₂ through decomposition of 2,5-furan-bis(iminoguanidine) carbonate tetrahydrate meets Jander model (n=3):

1−(1−α)^{1/3}=kt

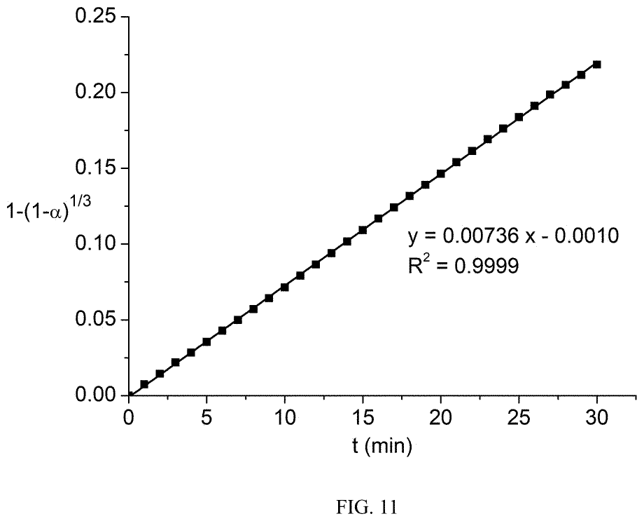

In the above formula, α=(m₀−m)/(m₀−m_{f}), m₀ is the initial weight and m_{f} is the final weight (see FIG. 11).

### Example 39: Differential Scanning Calorimetric Experiment and Analysis of 2,5-furan-bis(iminoguanidine) Carbonate Tetrahydrate

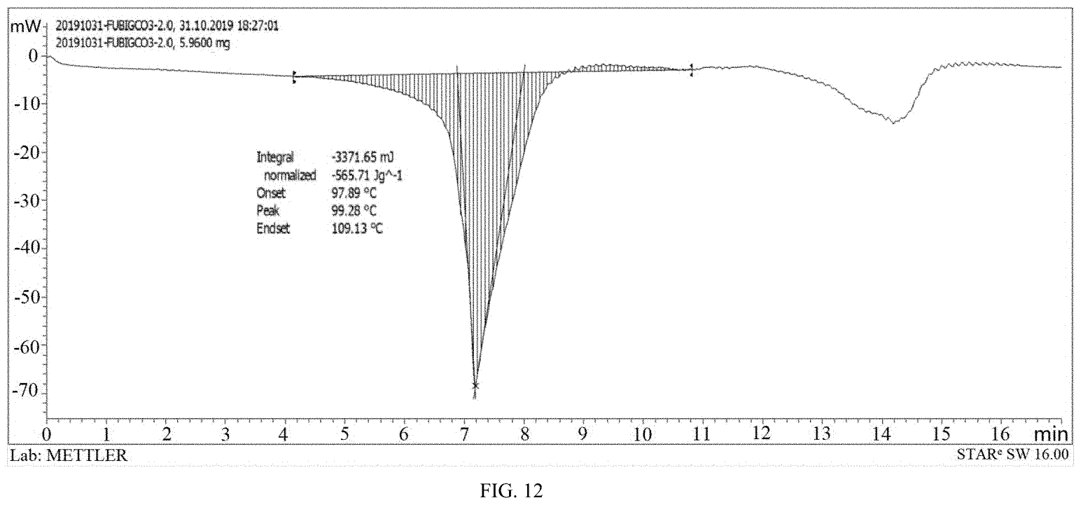

Differential scanning calorimetric analysis was carried out on 2,5-furan-bis(iminoguanidine) carbonate tetrahydrate, temperature range was 30-200° C., and the heating rate was 10 K/min. As shown in FIG. 12, the jump part within a range of 97.89-109.13° C. in the curve is a chemical process where 2,5-furan-bis(iminoguanidine) carbonate tetrahydrate releases carbon dioxide and water. Through the area of the integral curve, it can be seen that 2,5-furan-bis(iminoguanidine) carbonate tetrahydrate can absorb the heat of 565.71 J/g (namely 209.31 kJ/mol) when releasing carbon dioxide.

### Example 40: Determination of 2,5-furan-bis(iminoguanidine) pKa

0.077 g of FUBIG hydrochloride and 0.585 g of NaCl were weighed, the volume was metered to 50 mL with ultrapure water, and 26 μL of HCl (36%) was dropwise added. The mixed solution was stirred at 25° C. until the temperature was stabilized. The pH meter was calibrated respectively using potassium hydrogen phthalate (pH 4.00) buffer, mixed phosphate buffer (pH 6.86), sodium tetraborate buffer (pH 9.18), and then titration was conducted with 0.1M NaOH solution. After each titration, the system was stabilized after waiting for 7min, and then the next titration was conducted. The volume and pH value of each titration were recorded.

Plot was made according to the ph-V (mL). By combining one variable differential and two variable differential, pKₐ₁=7.57 and pKₐ₂=8.71 were solved. The same method was used to obtain: 15° C., pKₐ₁=7.79, pKₐ₂=8.90; at 20° C., pKₐ₁=7.68, pKₐ₂=8.82; at 30° C., pKₐ₁=7.49, pKₐ₂=8.66; at 35° C., pKₐ₁=7.31, pKₐ₂=8.59 (as shown in Table 3).

TABLE 3
Determination of pKₐ value of FuBIG (15-35° C.)

| Temperature(° C.) | pKₐ₁ | pKₐ₂ |
|---|---|---|
| 15 | 7.79 | 8.90 |
| 20 | 7.68 | 8.82 |
| 25 | 7.57 | 8.71 |
| 30 | 7.49 | 8.66 |
| 35 | 7.31 | 8.59 |

### Example 41: Determination of Solubility Product of 2,5-furan-bis(iminoguanidine) Carbonate Tetrahydrate (FuBIGH₂(CO₃)(H₂O)₄) and Solubility of 2,5-furan-bis(iminoguanidine) Hydrochloride, Nitrate, Sulfite and Sulfate

(1) Preparation of 2,5-furan-bis(iminoguanidine) carbonate

5 ml of 0.05 M 2,5-furan-bis(iminoguanidine) hydrochloride aqueous solution and 5 ml of 0.05 M sodium bicarbonate aqueous solution were weighed, added into a reaction bottle, stirred for 2 hours, filtered at reduced pressure and dried to obtain 2,5-furan-bis(iminoguanidine) carbonate whose weight was 60 mg.

(2) Preparation of 2,5-furan-bis(iminoguanidine) sulfate

5 ml of 0.05 M 2,5-furan-bis(iminoguanidine) hydrochloride aqueous solution and 5 ml of 0.05 M anhydrous sodium sulfate aqueous solution were weighed, added into a reaction flask, stirred for 2 h, filtered at reduced pressure and dried to obtain 2,5-furan-bis(iminoguanidine) sulfate whose weight was 30 mg.

(3) Preparation of 2,5-furan-bis(iminoguanidine) nitrate

5 ml of 0.05 M 2,5-furan-bis(iminoguanidine) hydrochloride aqueous solution and 5 ml of 0.1 M sodium nitrate aqueous solution were weighed, added into a reaction flask, stirred for 2 h, filtered at reduced pressure and dried to obtain 2,5-furan-bis(iminoguanidine) nitrate whose weight was 68 mg.

(4) Preparation of 2,5-furan-bis(iminoguanidine) sulfite

5 ml of 0.05 M 2,5-furan-bis(iminoguanidine) hydrochloride aqueous solution and 5 ml of 0.05 M anhydrous sodium sulfite aqueous solution were weighed, added into a reaction bottle, stirred for 2 h, filter at reduced pressure and dried to obtain 2,5-furan-bis(iminoguanidine) sulfite whose weight was 59 mg.

(5) Preparation of 2,5-furan-bis(iminoguanidine) phosphate

5 ml of 0.05 M 2,5-furan-bis(iminoguanidine) hydrochloride and 5 ml of 0.05 M disodium hydrogen phosphate were weighed, added into a reaction bottle, stirred for 2 hours, filtered at pressure and dried to obtain 2,5-furan-bis(iminoguanidine) phosphate whose weight was 60 mg.

(6) Preparation of 2,5-furan-bis(iminoguanidine) sulfide

5 ml of 0.05 M 2,5-furan-bis(iminoguanidine) hydrochloride and 5 ml of 0.05 M sodium hydrosulfide were weighed, added into a reaction flask, stirred for 2 hours, filtered at reduced pressure and dried to obtain 2,5-furan-bis(iminoguanidine) sulfide whose weight was 57 mg.

(7) Determination of solubility product of 2,5-furan-bis(iminoguanidine) carbonate tetrahydrate (FUBIGH₂(CO₃)(H₂O)₄) and water solubility of 2,5-furan-bis(iminoguanidine) hydrochloride, nitrate, sulfite, sulfate, phosphate and sulfide.

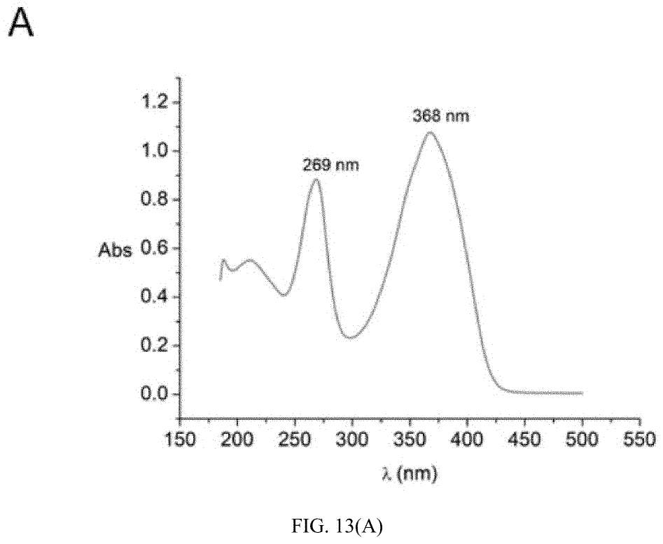

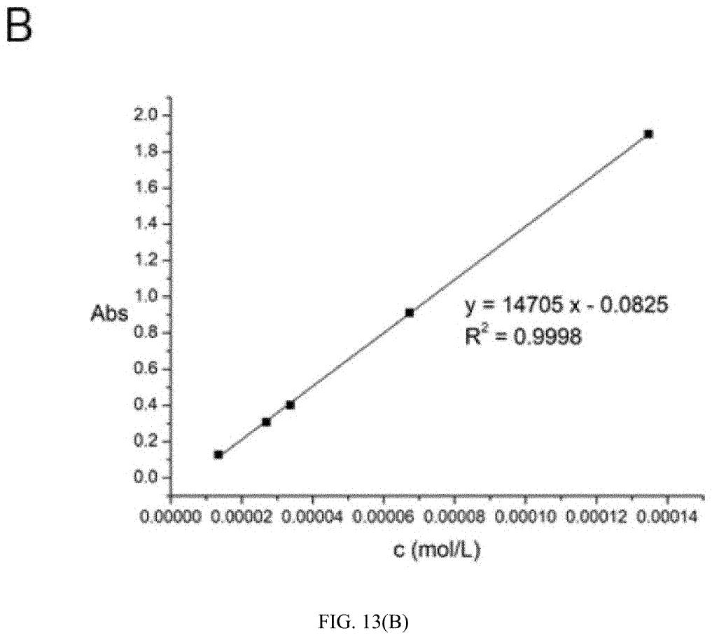

The 2,5-furan-bis(iminoguanidine) hydrochloride aqueous solutions having the concentrations of 1.35×10⁻⁵ M, 2.69×10⁻⁵ M, 3.36×10⁻⁵ M, 6.73×10⁻⁵ M and 1.35×10⁻⁴ M were respectively prepared and diluted. The ultraviolet spectrum scanning (200-700 nm) was conducted with 2,5-furan-bis(iminoguanidine) hydrochloride aqueous solution having the concentration of 3.36×10⁻⁵ M to obtain the maximum absorption wavelength λₘₐₓ=368 nm (as shown in FIG. 13A). The absorbance of the above concentration gradients was determined under this wavelength, and standard curves were plotted to obtain a curve equation Y=14705×−0.0825 (as shown in FIG. 13B).

The aqueous solutions of saturated 2,5-furan-bis(iminoguanidine) carbonate tetrahydrate (FUBIGH₂ (CO₃)(H₂O)₄) aqueous solution, saturated 2,5-furan-bis(iminoguanidine) nitrate aqueous solution, 2,5-furobiimidine sulfite solution, 2,5-furan-bis(iminoguanidine) hydrochloride solution, saturated 2,5-furan-bis(iminoguanidine) phosphate aqueous solution and saturated 2,5-furan-bis(iminoguanidine) sulfide aqueous solution were respectively prepared and diluted by 100 times, 1000 times, 100 times, 10000 times, 100 times and 100 times respectively. The ultraviolet absorbance was determined to be substituted into the equation Y=14705×−0.0825, so as to obtain the concentrations of 9.3 (4)×10⁻³M, 3.3 (2)×10⁻²M, 1.4 (2)×10⁻³M, 0.74 (6) M, 3.1 (3)×10⁻³M, 4.7 (4)×10⁻³M (as shown in Table 4). The pH of saturated 2,5-furan-bis(iminoguanidine) carbonate tetrahydrate (FUBIGH₂ (CO₃)(H₂O)₄) aqueous solution was determined as 8.48 by a pH meter. It was known that the pKa of HCO₃⁻ was 10.32. In the above case, FUBIG pKₐ₁=7.57 and pKₐ₂=8.71 were calculated. The concentration of carbonate ion [CO₃²⁻] in the saturated 2,5-furan-bis(iminoguanidine) tetrahydrate (FuBIGH₂(CO₃)(H₂O)₄) was 1.3(3)×10⁻⁴ M, and the concentration of 2,5-furan-bis(iminoguanidine) dihydrogen ion [FuBIGH₂²⁺] was 6.72×10⁻⁴M. According to the solubility product formula, the solubility product of 2,5-furan-bis(iminoguanidine) carbonate tetrahydrate (FuBIGH₂(CO₃)(H₂O)₄) was as follows:

Kₛₚ=γ₊² [FuBIGH₂²⁺][CO₃²⁻]=0.828² [6.72×10⁻⁴][1.33×10⁻⁴]=6.1(3)×10⁻⁸

TABLE 4
Solubility of various FuBIG salts

| | Solubility(FuBIG cation |
|---|---|
| Sample(anions) | concentration, M) |
| Carbonate | 9.3 (4) × 10⁻³ |
| Nitrate | 3.3 (2) × 10⁻² |
| Sulfite | 1.4 (2) × 10⁻³ |
| Hydrochloride | 0.74 (6) |
| Phosphate | 3.1 (2) × 10⁻³ |
| Sulfide | 4.7 (4) × 10⁻³ |

The above experiments show that the anionic salts of 2,5-furan-bis(iminoguanidine) have low water solubility except hydrochloride and can be separated from the aqueous phase, confirming that 2,5-furan-bis(iminoguanidine) can be used as an anion precipitant.

According to the above embodiments and related literatures, all physical and chemical data of the process for absorbing and releasing carbon dioxide by 2,5-furan-bis(iminoguanidine) can be summarized in Table 5.

TABLE 5
Equilibrium constants (K) and enthalpy changes (ΔH) of various
reactions in the process of absorbing and releasing carbon dioxide by
2,5-furan-bis(iminoguanidine)

| | | Equilibrium | Enthalpy change |
|---|---|---|---|
| Number | Reaction process | constant K | ΔH (kJ/mol) |
| 1 | FuBIG dissolution | 4.03 × 10⁻¹ | 49.19 |
| 2 | FuBIG protonation | 1.91 × 10⁻¹² | 46.08 |
| 3 | CO₂ dissolution | 3.4 × 10⁻² | −19.4 |
| 4 | Generation of HCO₃⁻ | 3.02 × 10⁷ | −50 |
| 5 | Generation of CO₃²⁻ | 4.66 × 10³ | −40.4 |
| 6 | FuBIGH₂(CO₃)(H₂O)₄ | 1.63 × 10⁷ | −101.57 |
| | precipitate | | |
| 7 | Total CO₂ absorbing process | 5.97 × 10⁴ | −116.1 |
| 8 | Total CO₂ releasing process | — | 209.31 |

What is claimed is:

1. A furan skeleton-containing iminoguanidine derivative, which means 2,5-furan-bis(iminoguanidine) and derivatives thereof, and comprises 2,5-furan-bis(iminoguanidine) and acceptable salts of 2,5-furan-bis(iminoguanidine) as well as solvates thereof.

2. The furan skeleton-containing iminoguanidine derivative according to claim 1, wherein the 2,5-furan-bis(iminoguanidine) has the following structural formula:

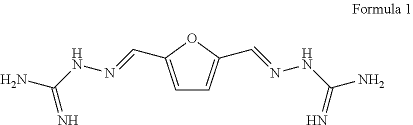 [SMILES: N=C(N)N/N=C/c1ccc(/C=N/NC(=N)N)o1]

3. The furan skeleton-containing iminoguanidine derivatives according to claim 1, wherein the acceptable salts of 2,5-furan-bis(iminoguanidine) as well as solvates thereof comprise carbonate and solvates thereof, sulfite and solvates thereof, sulfide and solvates thereof, hydrochloride and solvates thereof, sulfate and solvates, nitrate and solvates thereof, phosphate and solvates thereof, hypochlorite and solvates thereof, perchlorate and solvates thereof, bichromate and solvates thereof, and permanganate and solvates thereof.

4. A solvate of a salt of 2,5-furan-bis(iminoguanidine) according to claim 3, wherein the solvate is 2,5-furan-bis(iminoguanidine) carbonate tetrahydrate which has the following structural formula:

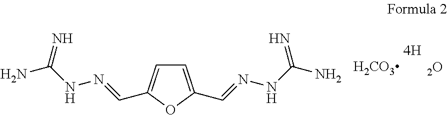

5. A method for preparing the 2,5-furan-bis(iminoguanidine) according to claim 2, comprising the following steps: allowing 2,5-diformylfuran as a raw material to react with aminoguanidine hydrochloride in solvent A; after the reaction is ended, allowing the reaction solution to be subjected to standing at a certain temperature, and filtering to obtain 2,5-furan-bis(iminoguanidine) hydrochloride; and alkalizing and secondarily standing to obtain 2,5-furan-bis(iminoguanidine), wherein the 2,5-diformylfuran is prepared by using renewable biomass resource 5-hydroxymethylfurfural as a raw material; the molar ratio of 2,5-diformylfuran to aminoguanidine hydrochloride is 1:11:3; the molar ratio of 2,5-furan-bis(iminoguanidine) hydrochloride to alkali is 1:21:4; the solvent A is methanol, ethanol, 1,4-dioxane or tetrahydrofuran; the alkali adopted by alkalizing is sodium hydroxide, potassium hydroxide, sodium carbonate, potassium carbonate or cesium carbonate.

6. Use of the 2,5-furan-bis(iminoguanidine) according to claim 1 as an acidic gas absorbent, wherein in solvent B, 2,5-furan-bis(iminoguanidine) is in contact with an acidic gas or a mixed gas containing the acidic gas to form a precipitate to be separated out, and filtered to obtain precipitate A, namely, a salt containing 2,5-furan-bis(iminoguanidine) and anions related to the acidic gas; the salt has extremely low liquid phase solubility, can release the acidic gas when being heated to a certain temperature and allows 2,5-furan-bis(iminoguanidine) to be regenerated and recycled.

7. Use of the 2,5-furan-bis(iminoguanidine) according to claim 6 as an acidic gas absorbent, wherein the acidic gas comprises carbon dioxide, sulfur dioxide, sulfur trioxide, nitrogen dioxide, nitric oxide, nitrous oxide or hydrogen sulfide; the mixed gas containing the acidic gas is obtained by mixing the above acidic gases in any ratios and by mixing one or more acidic gases with air, nitrogen, oxygen or inert gas in any ratios; the solvent B is one or a mixture of more of water, methanol, ethanol, acetone, tetrahydrofuran, acetonitrile, 1,4-dioxane, sulfolane, N-methylpyrrolidone, poly(glycol dimethyl ether) or propylene carbonate.

8. Use of the 2,5-furan-bis(iminoguanidine) according to claim 6 as the acidic gas absorbent, wherein the 2,5-furan-bis(iminoguanidine), as a carbon dioxide absorbent, is used for capture, utilization and storage of carbon dioxide.

9. Use of the 2,5-furan-bis(iminoguanidine) derivative according to claim 1 as an anionic precipitant, wherein in the solvent B, 2,5-furan-bis(iminoguanidine) generates a strong bonding effect with anions and forms a precipitate to be separated out; the anion comprises carbonate, bicarbonate, sulfite, sulfate, hydrogen sulfate, nitrate, hydrogen sulfide, phosphate, hydrogen phosphate, dihydrogen phosphate, perchlorate, hypochlorite, bichromate or permanganate.

10. Use of the 2,5-furan-bis(iminoguanidine) derivative according to claim 1 in a reaction where carbon dioxide participates, wherein the 2,5-furan-bis(iminoguanidine) derivative is used as a carbon dioxide gas source.

11. A 2,5-furandiiminoguanidine carbonate tetrahydrate crystal, wherein the crystal is a 2,5-furandiiminoguanidine carbonate tetrahydrate crystal formed from 2,5-furandiiminoguanidine according to claim 1, carbon dioxide and water; structural analysis is carried out via single crystal X-ray diffraction to obtain a triclinic system, wherein space group is P-1, and cell parameters are as follows: a=11.1919(4) Å, B=12.7930(5) Å, C=14.1784(4) Å, α=97.547(3)°, β=111.174(3)°, and γ=112.709(4)°; cell volume=1657.46 (11) Å³; density=1.484 g/cm³.

12. A crystal form of 2,5-furandiiminoguanidine carbonate tetrahydrate, wherein the crystal form is a crystal form of 2,5-furandiiminoguanidine carbonate tetrahydrate formed from 2,5-furandiiminoguanidine of claim 1, carbon dioxide and water; via X-ray powder diffraction represented by 2θ angle and interplanar crystal spacing (d value), the crystal has characteristic peaks at about 6.85 (12.9), 7.87 (11.2), 8.67 (10.2), 13.48 (6.6), 15.27 (5.8), 15.87 (5.6), 19.05 (4.7), 19.77 (4.5), 21.06 (4.2), 24.45 (3.6), 25.75 (3.5), 27.75 (3.5) and 27.98 (3.2).
